(19) United States
(12) Patent Application Publication
(10) Pub. No.: US 2017/0224544 A1
(43) Pub. Date: Aug. 10, 2017

(54) ABSORBENT ARTICLE WITH EXUDATE INDICATOR

(71) Applicant: Kimberly-Clark Worldwide, Inc., Neenah, WI (US)

(72) Inventors: Kristen Alene Decker, Grafton, WI (US); Andrew Thomas Hammond, Appleton, WI (US); Davis Dang Hoang Nhan, Appleton, WI (US); Michael John Niemeyer, Appleton, WI (US); Wing-Chak Ng, Roswell, GA (US); Christine Marie Kotula, Woodstock, GA (US)

(21) Appl. No.: 15/502,227

(22) PCT Filed: Aug. 28, 2014
(86) PCT No.: PCT/US2014/053059

§ 371 Date: Feb. 7, 2017

(51) Int. Cl. A61F 13/42; A61F 13/495; B29C 47/00; B29C 47/06; B29C 65/00; A61F 13/49; A61F 13/15

(52) U.S. Cl. CPC A61F 13/42; A61F 13/49011; A61F 13/495; A61F 13/15617; B29C 47/065; B29C 66/7294; B29C 47/0064; A61F 2013/1513; A61F 2013/15284; A61F 2013/49092; B29K 2995/0092; B29K 2995/0025; B29L 2031/4878

(57) ABSTRACT

An absorbent article (10, 110, 210) can include an absorbent assembly (43) including a bodyside liner (28), an outer cover (26, 126, 226), and an absorbent body (35) disposed between the bodyside liner (28) and the outer cover (26, 126, 226). The outer cover (26, 126, 226) can include at least one opaque region (64, 164, 264) and a first indicator region (66, 166, 266). The first indicator region (66, 166, 266) can have a light transmittance that is greater than a light transmittance of the at least one opaque region (64, 164, 264). At least a portion of the first indicator region (66, 166, 266) can be located between the first longitudinal edge (36) of the absorbent body (35) and the first coupling length (45) of the first containment flap (44) in at least the crotch region (16) of the absorbent article (10, 110, 210).

### TECHNICAL FIELD

The present invention relates to absorbent articles.

### BACKGROUND OF THE DISCLOSURE

One of the primary functions of personal care absorbent articles is to retain and absorb body exudates such as urine, fecal material, blood, and menses. Disposable absorbent articles are typically designed in such a fashion to hide such body exudates once they are retained and absorbed by the article. For example, some disposable absorbent articles, such as diapers, typically utilize an outer cover that has a high degree of opacity such that the absorbent core, and the bodily exudates absorbed thereby, cannot be seen through the outer cover of the diaper. Such an outer cover configuration can also be employed for the purposes of providing an outer cover with an appealing uniform color, such as white, that in some circumstances, can serve as a neutral background color to be printed upon with colored outer cover graphics. While such outer cover configurations protect against the viewing of such bodily exudates through the absorbent article while the article is being worn, the timing of a wearer's exudate secretions are not often known by the caregiver.

Continued wearing of an absorbent article after exudates have been insulted onto the absorbent article can lead to a few different undesirable effects. For example, prolonged wearing of the absorbent article that is retaining body exudates can increase the potential for skin irritation. In addition, continued wearing of such an absorbent article can also lead to an increased likelihood that the gasketing systems of the absorbent article become compromised. These undesirable effects can be more common with respect to semi-solid fecal material, such as low viscosity fecal material which can be prevalent with younger children. Such fecal material can have difficulty penetrating the bodyside liner of the absorbent article, and tend to spread across the surface of the bodyside liner.

Some absorbent articles include indicators on the outer cover that can provide an indication of wetness within the absorbent article when the indicators become wetted. However, such indicators are generally disposed between the outer cover and the absorbent core, and thus, only provide a caregiver with an indication that the diaper is retaining body exudates when the exudates are present between the absorbent core and the outer cover. Based on this configuration, some bodily exudates, such as fecal material, may not trigger such wetness indicators for some time, and thus, may not signal to a caregiver that the absorbent article is retaining body exudates. Additionally, these wetness indicators, as well as other exudate indicators in absorbent articles, are generally aligned with the longitudinal axis of the absorbent article, a position that is difficult for a caregiver to view or access when the wearer has clothing covering the absorbent article.

Thus, there remains a need for an absorbent article that can provide a caregiver an indication that the absorbent article is retaining exudates that does not rely on wetness. There also remains a need for an absorbent article that includes an indicator that provides easier viewing access for a caregiver when the wearer has clothing covering the absorbent article.

### SUMMARY OF THE DISCLOSURE

In one embodiment, the present invention provides an absorbent article including a front waist region, a rear waist region, and a crotch region. The absorbent article can include an absorbent assembly including a bodyside liner, an outer cover, and an absorbent body disposed between the bodyside liner and the outer cover. The absorbent body can include a first longitudinal edge and a second longitudinal edge. The absorbent article can further include a pair of containment flaps including a first containment flap and a second containment flap. The first containment flap can be coupled to the absorbent assembly along a first coupling length and the second containment flap can be coupled to the absorbent assembly along a second coupling length. The outer cover can include at least one opaque region and a first indicator region. The first indicator region can include a light transmittance that is greater than a light transmittance of the at least one opaque region. At least a portion of the first indicator region can be located between the first longitudinal edge of the absorbent body and the first coupling length of the first containment flap in at least the crotch region.

In another embodiment, the present invention provides an absorbent article including a front waist region, a rear waist region, and a crotch region. The absorbent article can include an absorbent assembly including a bodyside liner, an absorbent body including a first longitudinal edge and a second longitudinal edge, and an outer cover. The absorbent body can be disposed between the bodyside liner and the outer cover. The outer cover can include a substantially continuous film including at least one opaque area corresponding to a first opaque region of the outer cover and a first indicator area corresponding to a first indicator region of the outer cover. The first indicator region can include a light transmittance that is greater than a light transmittance of the at least one opaque region and can be located in at least the crotch region. The outer cover can be configured such that a thickness of the opaque area of the film and a thickness of the first indicator area of the film are substantially similar.

In yet another embodiment, the present invention provides a method of manufacturing an outer cover for an absorbent article, where the outer cover includes a segmented film. The method can include providing a first polymer material and a second polymer material and providing an opacifying filler material. The method can further include providing an extrusion die. Additionally, the method can include extruding the first polymer material and the opacifying filler material through the extrusion die to form a first opaque area and a second opaque area. The method can include coextruding the second polymer material through the extrusion die to form a first indicator area between the first opaque area and the second opaque area to form the segmented film. The first indicator area can include a light transmittance that is greater than a light transmittance of the first opaque area and greater than a light transmittance of the second opaque area.

### BRIEF DESCRIPTION OF DRAWINGS

A full and enabling disclosure thereof, directed to one of ordinary skill in the art, is set forth more particularly in the remainder of the specification, which makes reference to the appended figures in which:

FIG. 1 is a side perspective view of an exemplary embodiment of an absorbent article, such as a diaper, in a fastened condition.

FIG. 2 is a top plan view of the absorbent article of FIG. 1 in an unfastened, stretched, and laid flat condition with the body facing surface of the absorbent article which contacts the wearer facing the viewer, portions of the absorbent article being cut away for clarity of illustration.

FIG. 3 is an exploded, cross-sectional view of the absorbent article of FIG. 2 taken along line 3-3.

FIG. 4 is a top plan view of another exemplary embodiment of an absorbent article in an unfastened, stretched, and laid flat condition with the body facing surface of the absorbent article which contacts the wearer facing the viewer, portions of the absorbent article being cut away for clarity of illustration.

FIG. 5 is an exploded, cross-sectional view of the absorbent article of FIG. 4 taken along line 5-5.

FIG. 6 is a top plan view of yet another exemplary embodiment of an absorbent article in an unfastened, stretched, and laid flat condition with the body facing surface of the absorbent article which contacts the wearer facing the viewer, portions of the absorbent article being cut away for clarity of illustration.

FIG. 7 is an exploded, cross-sectional view of the absorbent article of FIG. 6 taken along line 7-7.

FIG. 8 is a perspective view of a device used in the Light Transmittance Test.

FIG. 9 is a detailed perspective view of the sampling port of the device of FIG. 8.

FIG. 10 is a top plan view of a portion of a sample of an inner layer of an outer cover including an indicator area and two opaque areas.

FIG. 11 is a top plan view of the portion of the sample of an inner layer of an outer cover from FIG. 10 held over the sampling port of the device of FIG. 8 to be measured in the Light Transmittance Test.

Repeat use of reference characters in the present specification and drawings is intended to represent the same or analogous features or elements of the disclosure.

### DETAILED DESCRIPTION OF THE DISCLOSURE

In an embodiment, the present disclosure is generally directed towards an absorbent article having an outer cover that includes at least one indicator region and at least one opaque region, with an opacity of the indicator region being less than an opacity of the opaque region. In preferred embodiments, the selective location of the indicator region can provide an accessible indicator for caregivers as to whether the article is retaining exudates, and in particular, fecal material. Each example is provided by way of explanation and is not meant as a limitation. For example, features illustrated or described as part of one embodiment or figure can be used on another embodiment or figure to yield yet another embodiment. It is intended that the present disclosure include such modifications and variations.

When introducing elements of the present disclosure or the preferred embodiment(s) thereof, the articles “a”, “an”, “the” and “said” are intended to mean that there are one or more of the elements. The terms “comprising”, “including” and “having” are intended to be inclusive and mean that there may be additional elements other than the listed elements. Many modifications and variations of the present disclosure can be made without departing from the spirit and scope thereof. Therefore, the exemplary embodiments described above should not be used to limit the scope of the invention.

### Definitions

The term “absorbent article” refers herein to an article which may be placed against or in proximity to the body (i.e., contiguous with the body) of the wearer to absorb and contain various liquid, solid, and semi-solid exudates discharged from the body. Such absorbent articles, as described herein, are intended to be discarded after a limited period of use instead of being laundered or otherwise restored for reuse. It is to be understood that the present disclosure is applicable to various disposable absorbent articles, including, but not limited to, diapers, training pants, youth pants, swim pants, feminine hygiene products, including, but not limited to, menstrual pads, incontinence products, medical garments, surgical pads and bandages, other personal care or health care garments, and the like without departing from the scope of the present disclosure.

The term “acquisition layer” refers herein to a layer capable of accepting and temporarily holding liquid body exudates to decelerate and diffuse a surge or gush of the liquid body exudates and to subsequently release the liquid body exudates therefrom into another layer or layers of the absorbent article.

The term “bonded” refers herein to the joining, adhering, connecting, attaching, or the like, of two elements. Two elements will be considered bonded together when they are joined, adhered, connected, attached, or the like, directly to one another or indirectly to one another, such as when each is directly bonded to intermediate elements. The bonding of one element to another can occur via continuous or intermittent bonds.

The term “carded web” refers herein to a web containing natural or synthetic staple length fibers typically having fiber lengths less than about 100 mm. Bales of staple fibers can undergo an opening process to separate the fibers which are then sent to a carding process which separates and combs the fibers to align them in the machine direction after which the fibers are deposited onto a moving wire for further processing. Such webs are usually subjected to some type of bonding process such as thermal bonding using heat and/or pressure. In addition to or in lieu thereof, the fibers may be subject to adhesive processes to bind the fibers together such as by the use of powder adhesives. The carded web may be subjected to fluid entangling, such as hydroentangling, to further intertwine the fibers and thereby improve the integrity of the carded web. Carded webs, due to the fiber alignment in the machine direction, once bonded, will typically have more machine direction strength than cross machine direction strength.

The term “film” refers herein to a thermoplastic film made using an extrusion and/or forming process, such as a cast film or blown film extrusion process. The term includes apertured films, slit films, and other porous films which constitute liquid transfer films, as well as films which do not transfer fluids, such as, but not limited to, barrier films, filled films, breathable films, and oriented films.

The term “gsm” refers herein to grams per square meter.

The term “hydrophilic” refers herein to fibers or the surfaces of fibers which are wetted by aqueous liquids in contact with the fibers. The degree of wetting of the materials can, in turn, be described in terms of the contact angles and the surface tensions of the liquids and materials involved. Equipment and techniques suitable for measuring the wettability of particular fiber materials or blends of fiber materials can be provided by Cahn SFA-222 Surface Force Analyzer System, or a substantially equivalent system. When measured with this system, fibers having contact angles less than 90 are designated “wettable” or hydrophilic, and fibers having contact angles greater than 90 are designated “nonwettable” or hydrophobic.

The term “light transmittance” or “light transmission” refers herein to a measured property of a substrate, or substrates, as defined by the Light Transmittance Test further described herein.

The term “liquid impermeable” refers herein to a layer or multi-layer laminate in which liquid body exudates, such as urine, will not pass through the layer or laminate, under ordinary use conditions, in a direction generally perpendicular to the plane of the layer or laminate at the point of liquid contact.

The term “liquid permeable” refers herein to any material that is not liquid impermeable.

The term “meltblown” refers herein to fibers formed by extruding a molten thermoplastic material through a plurality of fine, usually circular, die capillaries as molten threads or filaments into converging high velocity heated gas (e.g., air) streams which attenuate the filaments of molten thermoplastic material to reduce their diameter, which can be a microfiber diameter. Thereafter, the meltblown fibers are carried by the high velocity gas stream and are deposited on a collecting surface to form a web of randomly dispersed meltblown fibers. Such a process is disclosed, for example, in U.S. Pat. No. 3,849,241 to Butin et al., which is incorporated herein by reference. Meltblown fibers are microfibers which may be continuous or discontinuous, are generally smaller than about 0.6 denier, and may be tacky and self-bonding when deposited onto a collecting surface.

The term “nonwoven” refers herein to materials and webs of material which are formed without the aid of a textile weaving or knitting process. The materials and webs of materials can have a structure of individual fibers, filaments, or threads (collectively referred to as “fibers”) which can be interlaid, but not in an identifiable manner as in a knitted fabric. Nonwoven materials or webs can be formed from many processes such as, but not limited to, meltblowing processes, spunbonding processes, carded web processes, etc.

The term “pliable” refers herein to materials which are compliant and which will readily conform to the general shape and contours of the wearer's body.

The term “spunbond” refers herein to small diameter fibers which are formed by extruding molten thermoplastic material as filaments from a plurality of fine capillaries of a spinnerette having a circular or other configuration, with the diameter of the extruded filaments then being rapidly reduced by a conventional process such as, for example, eductive drawing, and processes that are described in U.S. Pat. No. 4,340,563 to Appel et al., U.S. Pat. No. 3,692,618 to Dorschner et al., U.S. Pat. No. 3,802,817 to Matsuki et al., U.S. Pat. Nos. 3,338,992 and 3,341,394 to Kinney, U.S. Pat. No. 3,502,763 to Hartmann, U.S. Pat. No. 3,502,538 to Peterson, and U.S. Pat. No. 3,542,615 to Dobo et al., each of which is incorporated herein in its entirety by reference. Spunbond fibers are generally continuous and often have average deniers larger than about 0.3, and in an embodiment, between about 0.6, 5 and 10 and about 15, 20 and 40. Spunbond fibers are generally not tacky when they are deposited on a collecting surface.

The term “superabsorbent” refers herein to a water-swellable, water-insoluble organic or inorganic material capable, under the most favorable conditions, of absorbing at least about 15 times its weight and, in an embodiment, at least about 30 times its weight, in an aqueous solution containing 0.9 weight percent sodium chloride. The superabsorbent materials can be natural, synthetic and modified natural polymers and materials. In addition, the superabsorbent materials can be inorganic materials, such as silica gels, or organic compounds, such as cross-linked polymers.

The term “thermoplastic” refers herein to a material which softens and which can be shaped when exposed to heat and which substantially returns to a non-softened condition when cooled.

The term “user” or “caregiver” refers herein to one who fits an absorbent article, such as, but not limited to, a diaper, training pant, youth pant, incontinent product, or other absorbent article about the wearer of one of these absorbent articles. A user and a wearer can be one and the same person.

Absorbent Article:

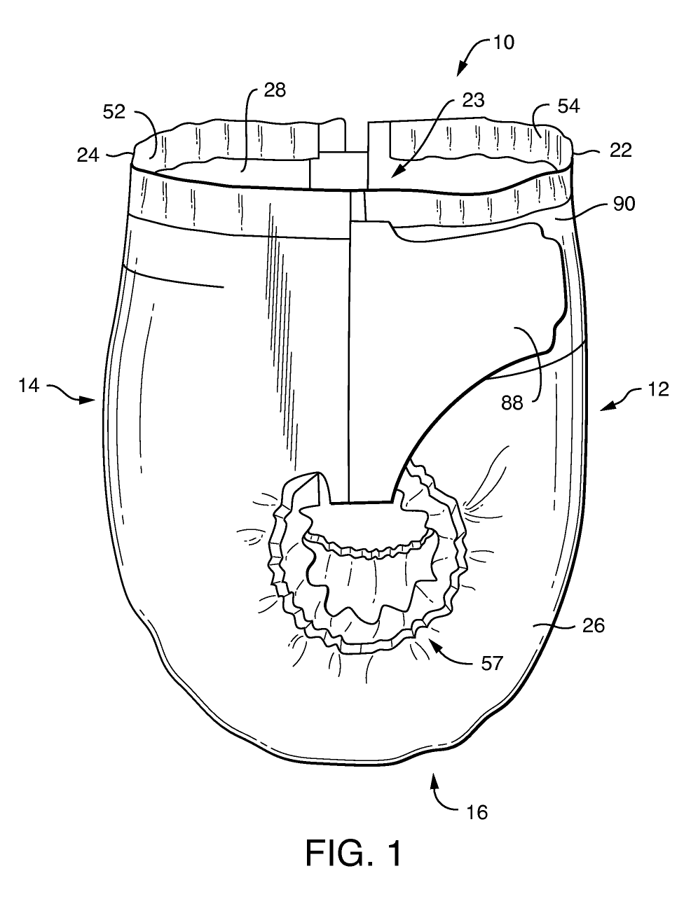

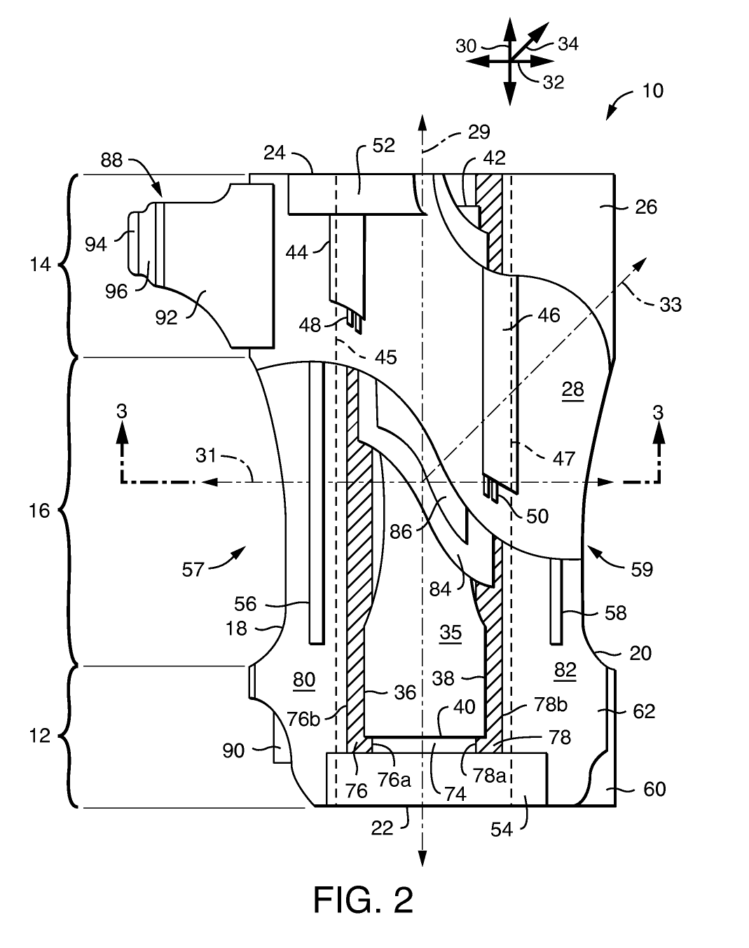

Referring to FIGS. 1 and 2, a non-limiting illustration of an absorbent article 10, for example, a diaper, is illustrated. Other embodiments of the absorbent article could include training pants, youth pants, adult incontinence garments, and feminine hygiene articles. While the embodiments and illustrations described herein may generally apply to absorbent articles manufactured in the product longitudinal direction, which is hereinafter called the machine direction manufacturing of a product, it should be noted that one of ordinary skill in the art could apply the information herein to absorbent articles manufactured in the latitudinal direction of the product, which hereinafter is called the cross direction manufacturing of a product, without departing from the spirit and scope of the disclosure.

The absorbent article 10 illustrated in FIGS. 1 and 2 includes a front waist region 12, a rear waist region 14, and a crotch region 16 disposed between the front waist region 12 and the rear waist region 14 and interconnecting the front and rear waist regions, 12, 14, respectively. The front waist region 12 can be referred to as the front end region, the rear waist region 14 can be referred to as the rear end region, and the crotch region 16 can be referred to as the intermediate region. The absorbent article 10 has a pair of longitudinal side edges, 18, 20, and a pair of opposite waist edges, respectively designated front waist edge 22 and rear waist edge 24. The front waist region 12 can be contiguous with the front waist edge 22 and the rear waist region 14 can be contiguous with the rear waist edge 24. The longitudinal side edges 18, 20 can extend from the front waist edge 22 to the rear waist edge 24.

The front waist region 12 can include the portion of the absorbent article 10 that, when worn, is positioned at least in part on the front of the wearer while the rear waist region 14 can include the portion of the absorbent article 10 that, when worn, is positioned at least in part on the back of the wearer. The crotch region 16 of the absorbent article 10 can include the portion of the absorbent article 10, that, when worn, is positioned between the legs of the wearer and can partially cover the lower torso of the wearer. The waist edges, 22 and 24, of the absorbent article 10 are configured to encircle the waist of the wearer and together define the central waist opening 23. Portions of the longitudinal side edges, 18 and 20, in the crotch region 16 can generally define leg openings when the absorbent article 10 is worn.

The absorbent article 10 can include an outer cover 26 and a bodyside liner 28. In an embodiment, the bodyside liner 28 can be bonded to the outer cover 26 in a superposed relation by any suitable means such as, but not limited to, adhesives, ultrasonic bonds, thermal bonds, pressure bonds, or other conventional techniques. The outer cover 26 can define a length in a longitudinal direction 30, and a width in the lateral direction 32, which, in the illustrated embodiment, can coincide with the length and width of the absorbent article 10. As illustrated in FIG. 2, the absorbent article 10 can have a longitudinal axis 29 extending in the longitudinal direction 30 and a lateral axis 31 extending in the lateral direction 32. The longitudinal axis 29 and the lateral axis 31 can define a horizontal plane. The absorbent article 10 can also have a vertical axis 33 extending in a vertical direction 34. The vertical axis 33 is perpendicular to the horizontal plane defined by the longitudinal axis 29 and the lateral axis 31.

FIG. 2 illustrates the absorbent article 10 with certain portions cut-away for illustrating additional aspects of the absorbent article 10. An absorbent body 35 can be disposed between the outer cover 26 and the bodyside liner 28. The absorbent body 35 can have longitudinal edges, 36 and 38, which, in an embodiment, can form portions of the longitudinal side edges, 18 and 20, respectively, of the absorbent article 10. The absorbent body 35 can have opposite end edges, 40 and 42, which, in an embodiment, can form portions of the waist edges, 22 and 24, respectively, of the absorbent article 10. In an embodiment, the absorbent body 35 can have a length and width that are the same as or less than the length and width of the absorbent article 10. The bodyside liner 28, the outer cover 26, and the absorbent body 35 can form part of an absorbent assembly 43. The absorbent article 10 can also include a fluid transfer layer 84 and a fluid acquisition layer 86, which can also form part of the absorbent assembly 43.

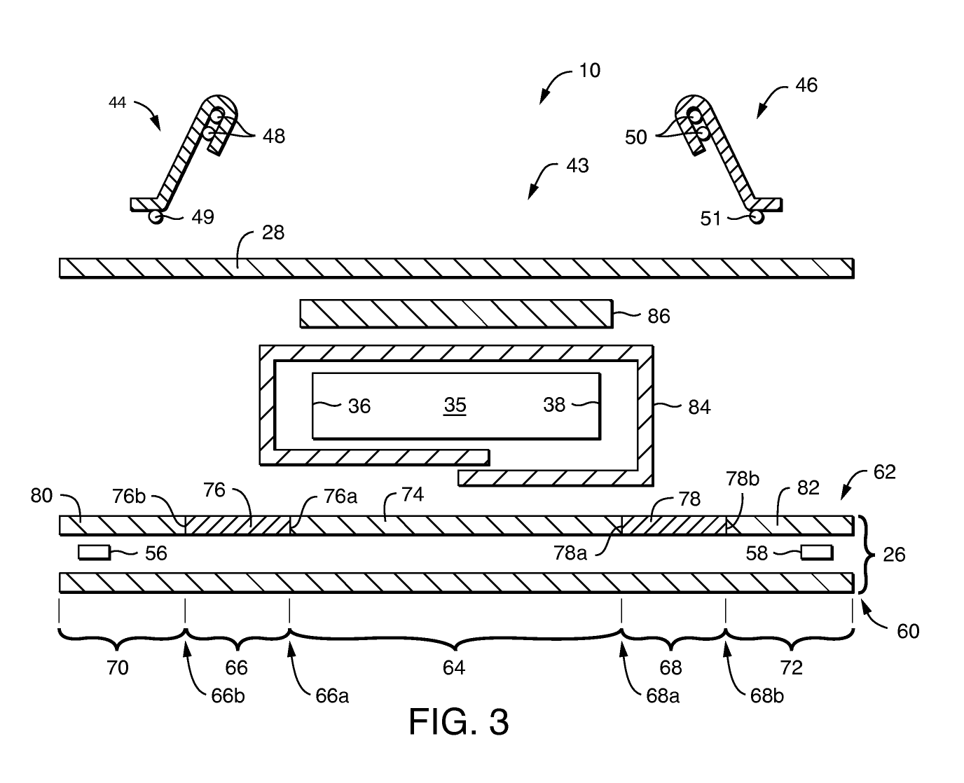

The absorbent article 10 can be configured to contain and/or absorb liquid, solid, and semi-solid body exudates discharged from the wearer. For example, containment flaps, 44 and 46, can be configured to provide a barrier to the lateral flow of body exudates. As illustrated in FIG. 2, each containment flap 44, 46 can include elastic members 48, 50. The elastic members 48, 50 can include one or more elastic strands (two are shown in FIG. 2) that are aligned substantially parallel to the longitudinal axis 29 of the absorbent article 10. The containment flaps 44, 46 are laterally spaced from one another, such that the containment flap 44 is on one side of the longitudinal axis 29 and the containment flap 46 is on an opposite side of the longitudinal axis 29. The containment flaps 44, 46 can be attached to the absorbent article 10 by being bonded to the absorbent assembly 43 along coupling lengths 45, 47, respectively. In the embodiment illustrated in FIG. 2, the containment flap 44 can be coupled to the absorbent assembly 43 by being bonded to the bodyside liner 28 along coupling length 45 with a bead of adhesive 49 (shown in FIG. 3). Similarly, the containment flap 46 can be coupled to the absorbent assembly 43 by being bonded to the bodyside liner 28 along coupling length 47 with a bead of adhesive 51 (shown in FIG. 3). The containment flaps, 44 and 46, can be located laterally inward from the longitudinal side edges, 18, 20 of the absorbent article 10, and can extend longitudinally along the entire length of absorbent article 10 or can extend partially along the length of the absorbent article 10. In the embodiment shown in FIG. 2, the containment flaps 44, 46 extend along the length of the absorbent article 10, and the coupling lengths 45, 47 are illustrated with a dashed line portraying their location along the length of the absorbent assembly 43.

It is to be noted that the containment flaps 44, 46 can be coupled to the absorbent assembly 43 other than by being bonded to the bodyside liner 28. As but one example, the containment flaps 44, 46 can alternatively be coupled to the absorbent assembly 43 by being bonded to the outer cover 26. Additionally, it is contemplated that the containment flaps 44, 46 can be coupled to the absorbent assembly 43 by means other than adhesives 49, 51. By way of example, the containment flaps 44, 46 could be bonded to the absorbent assembly 43 along coupling lengths 45, 47, respectively, by pressure bonding, ultrasonic bonding, stitching, or other means known to those in the art.

To further enhance containment and/or absorption of body exudates, in some embodiments the absorbent article 10 can suitably include a rear waist elastic member 52, a front waist elastic member 54, and leg elastic members, 56 and 58, as are known to those skilled in the art. The waist elastic members, 52 and 54, can be attached to the outer cover 26 and/or the bodyside liner 28 along the opposite waist edges, 24 and 22, and can extend over part or all of the waist edges, 24 and 22. In an embodiment shown in FIG. 2, the rear waist elastic member 52 is attached to the bodyside liner 28 and the containment flaps 44, 46 and the front waist elastic member 54 is attached to the outer cover 26. The leg elastic members, 56 and 58, can be attached to the outer cover 26 and/or the bodyside liner 28 along the opposite longitudinal side edges, 18 and 20, and positioned in the crotch region 16 of the absorbent article 10. The leg elastic members, 56 and 58, can be parallel to the longitudinal axis 29 or can be curved, as is known in the art.

Additional details regarding each of these elements of the absorbent article 10 described herein can be found below and with reference to the FIGS. 1 through 7.

Outer Cover:

The outer cover 26 and/or portions thereof can be breathable and/or liquid impermeable. The outer cover 26 and/or portions thereof can be elastic, stretchable, or non-stretchable. The outer cover 26 may be constructed of a single layer, multiple layers, laminates, spunbond fabrics, films, meltblown fabrics, elastic netting, microporous webs, bonded-carded webs or foams provided by elastomeric or polymeric materials. In an embodiment, for example, the outer cover 26 can be constructed of a microporous polymeric film, such as polyethylene or polypropylene.

In an embodiment, the outer cover 26 can be a single layer of a liquid impermeable material, such as a polymeric film. In an embodiment, the outer cover 26 can be suitably stretchable, and more suitably elastic, in at least the lateral direction 32 of the absorbent article 10. In an embodiment, the outer cover 26 can be stretchable, and more suitably elastic, in both the lateral 32 and the longitudinal 30 directions. In an embodiment, the outer cover 26 can be a multi-layered laminate in which at least one of the layers is liquid impermeable. In an embodiment, the outer cover 26 can be a two layer construction, including an outer layer 60 material and an inner layer 62 material which can be bonded together such as by a laminate adhesive. Suitable laminate adhesives can be applied continuously or intermittently as beads, a spray, parallel swirls, or the like, but it is to be understood that the inner layer 62 can be bonded to the outer layer 60 by other bonding methods, including, but not limited to, ultrasonic bonds, thermal bonds, pressure bonds, or the like.

The outer layer 60 of the outer cover 26 can be any suitable material and may be one that provides a generally cloth-like texture or appearance to the wearer. An example of such material can be a 100% polypropylene bonded-carded web with a diamond bond pattern available from Sandler A.G., Germany, such as 30 gsm Sawabond 4185® or equivalent. Another example of material suitable for use as an outer layer 60 of an outer cover 26 can be a 20 gsm spunbond polypropylene non-woven web. The outer layer 60 may also be constructed of the same materials from which the bodyside liner 28 can be constructed as described herein.

The liquid impermeable inner layer 62 of the outer cover 26 (or the liquid impermeable outer cover 26 where the outer cover 26 is of a single-layer construction) can be either vapor permeable (i.e., “breathable”) or vapor impermeable. The liquid impermeable inner layer 62 (or the liquid impermeable outer cover 26 where the outer cover 26 is of a single-layer construction) can be manufactured from a thin plastic film. The liquid impermeable inner layer 62 (or the liquid impermeable outer cover 26 where the outer cover 26 is of a single-layer construction) can inhibit liquid body exudates from leaking out of the absorbent article 10 and wetting articles, such as bed sheets and clothing, as well as the wearer and caregiver.

Where the outer cover 26 is of a single layer construction, it can be embossed and/or matte finished to provide a more cloth-like texture or appearance. The outer cover 26 can permit vapors to escape from the absorbent article 10 while preventing liquids from passing through. A suitable liquid impermeable, vapor permeable material can be composed of a microporous polymer film or a non-woven material which has been coated or otherwise treated to impart a desired level of liquid impermeability.

The outer cover 26 can include at least one opaque region 64 and at least one indicator region 66, 68. As shown in FIG. 3, the outer cover 26 can include a first indicator region 66 and a second indicator region 68. The first indicator region 66 can have a light transmittance that is greater than a light transmittance of the opaque region 64. Similarly, the second indicator region 68 can have a light transmittance that is greater than a light transmittance of the opaque region 64. In a preferred embodiment, the light transmittance of the first indicator region 66 can be substantially the same as the light transmittance of the second indicator region 68. The first indicator region 66 can be on a first side of the longitudinal axis 29 and the second indicator region 68 can be on a second side of the longitudinal axis 29. As shown in FIGS. 2 and 3, the first indicator region 66 can be separate from the second indicator region 68. In some embodiments, the first opaque region 64 can be disposed between the first indicator region 66 and the second indicator region 68. In some embodiments, the first opaque region 64 can be in a central, longitudinal portion of the absorbent article 10. As illustrated in FIGS. 2 and 3, the absorbent article 10 can also include opaque regions 70 and 72 that can be adjacent the respective longitudinal side edges 18, 20 of the absorbent article 10. The first indicator region 66 can be positioned between opaque region 70 and opaque region 64. The second indicator region can be positioned between opaque region 64 and opaque region 72.

As noted above, the outer cover 26 can include an inner layer 62, such as a polymeric film. As shown in FIGS. 2 and 3, the inner layer 62 can include a first opaque area 74 that corresponds to the opaque region 64 and indicator areas 80, 82 that correspond to indicator regions 70, 72 of the outer cover 26, respectively. The inner layer 62 can also include a first indicator area 76 that corresponds to the first indicator region 66 and a second indicator area 78 that corresponds to the second indicator region 68. The first indicator region 66 can include an inner longitudinal edge 66a and an outer longitudinal edge 66b. Similarly, the second indicator region 68 can include an inner longitudinal edge 68a and an outer longitudinal edge 68b. In the embodiment shown in FIGS. 2 and 3, the inner longitudinal edge 66a of the first indicator region 66 can correspond to the inner longitudinal edge 76a of the first indicator area 76 of the inner layer 62 and the outer longitudinal edge 66b of the first indicator region 66 can correspond to the outer longitudinal edge 76b of the first indicator area 76 of the inner layer 62. Likewise, the inner longitudinal edge 68a of the second indicator region 68 can correspond to the inner longitudinal edge 78a of the second indicator area 78 of the inner layer 62 and the outer longitudinal edge 68b of the second indicator region 68 can correspond to the outer longitudinal edge 78b of the second indicator area 78 of the inner layer 62.

The first indicator region 66 and the second indicator region 68 of the outer cover 26, and thus the first indicator area 76 and the second indicator area 78 of the inner layer 62 of the outer cover 26, can be selectively designed and located with respect to other components of the absorbent article 10 to provide benefits to a caregiver. The selective location of the indicator regions 66, 68 of the outer cover 26, and thus the first indicator area 76 and the second indicator area 78 of the inner layer 62 of the outer cover 26, as discussed throughout this disclosure is taken in the context of the absorbent article 10 being in the stretched, laid flat condition, such as that illustrated in FIGS. 2 and 3. For example, the first indicator region 66 can be selectively located in the outer cover 26 in at least the crotch region 16 (and in some embodiments in the front waist region 12 and the rear waist region 14) such that at least a portion of the first indicator region 66 is located between longitudinal edge 36 of the absorbent body 35 and the first coupling length 45 where the containment flap 44 is bonded to the absorbent assembly 43 (in the embodiment illustrated in FIGS. 2 and 3, the first coupling length 45 is located where the bead of adhesive 49 bonds the containment flap 44 to the bodyside liner 28). Stated in other words, the inner longitudinal edge 66a of the first indicator region 66 can be located internal to the first coupling length 45 (i.e., closer to the longitudinal axis 29 than the first coupling length 45 is to the longitudinal axis 29) and the outer longitudinal edge 66b of the first indicator region 66 can be located external to the longitudinal edge 36 of the absorbent body 35 (i.e., farther from the longitudinal axis 29 than the longitudinal edge 36 is to the longitudinal axis 29). In some embodiments, such as the embodiment illustrated in FIGS. 2 and 3, the entire lateral width of the first indicator region 66 is located between longitudinal edge 36 of the absorbent body 35 and the first coupling length 45 in the crotch region 16 of the absorbent article 10. The first indicator region 66 of the outer cover 26, and thus the first indicator area 76 of the inner layer 62, can extend from the front waist edge 22 to the rear waist edge 24.

The first indicator region 66 can also extend laterally inside of the longitudinal edge 36 of the absorbent body 35 in one or more of the front waist region 12, the rear waist region 14, and the crotch region 16. For example, in FIG. 2 the first indicator area 76 of the inner layer 62 of the outer cover 26 (and thus, the first indicator region 66 of the outer cover 26) extends laterally inside of the longitudinal edge 36 of the absorbent body 35 in the front waist region 12 such that the inner longitudinal edge 76a of the first indicator area 76 (and thus, the inner longitudinal edge 66a of the first indicator region 66) is closer to the longitudinal axis 29 than is the longitudinal edge 36 of the absorbent body 35.

The second indicator region 68 can be selectively designed in the outer cover 26 in a similar manner to the first indicator region 66 as described above. For example, the second indicator region 68 can be selectively designed in the outer cover 26 in at least the crotch region 16 (and in some embodiments in the front waist region 12 and the rear waist region 14) such that at least a portion of the second indicator region 68 is located between longitudinal edge 38 of the absorbent body 35 and the second coupling length 47 where the containment flap 46 is bonded to the absorbent assembly 43 (in the embodiment illustrated in FIGS. 2 and 3, the second coupling length 47 is located where the bead of adhesive 51 bonds the containment flap 46 to the bodyside liner 28). Stated in other words, the inner longitudinal edge 68a of the second indicator region 68 can be located internal to the second coupling length 47 (i.e., closer to the longitudinal axis 29 than the second coupling length 47 is to the longitudinal axis 29) and the outer longitudinal edge 68b of the second indicator region 68 can be located external to the longitudinal edge 38 of the absorbent body 35 (i.e., farther from the longitudinal axis 29 than the longitudinal edge 38 is to the longitudinal axis 29). In some embodiments, such as the embodiment illustrated in FIGS. 2 and 3, the entire lateral width of the second indicator region 68 is located between longitudinal edge 38 of the absorbent body 35 and the second coupling length 47 in the crotch region 16 of the absorbent article 10. The second indicator region 68 of the outer cover 26, and thus the second indicator area 78 of the inner layer 62, can extend from the front waist edge 22 to the rear waist edge 24.

The second indicator region 68 can also extend laterally inside of the longitudinal edge 38 of the absorbent body 35 in one or more of the front waist region 12, the rear waist region 14, and the crotch region 16. For example, in FIG. 2 the second indicator area 78 of the inner layer 62 of the outer cover 26 (and thus, the second indicator region 68 of the outer cover 26) extends laterally inside of longitudinal edge 36 in the front waist region 12 and the rear waist region 14 such that the inner longitudinal edge 78a of the second indicator area 78 (and thus, the inner longitudinal edge 68a of the second indicator region 68) is closer to the longitudinal axis 29 than is the longitudinal edge 38 of the absorbent body 35.

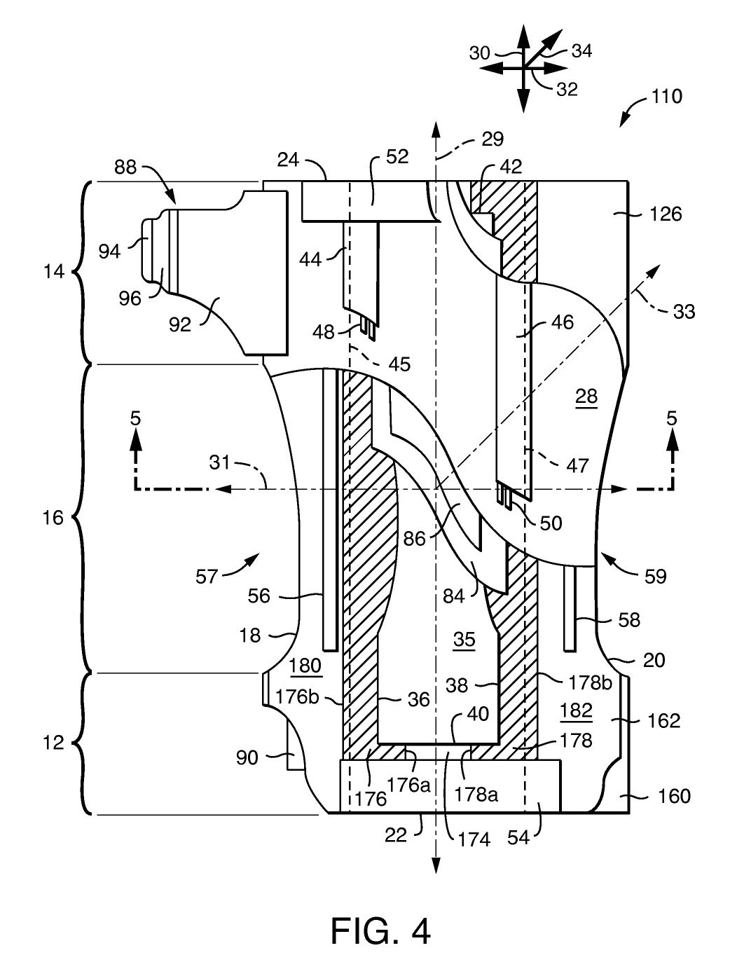

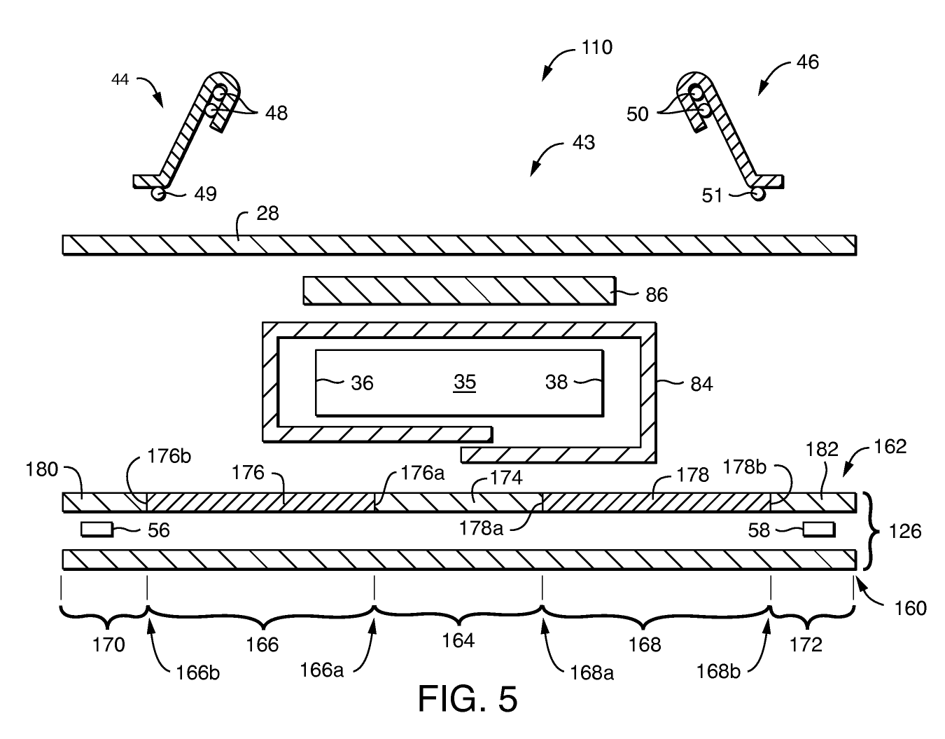

In another embodiment, such as that illustrated in FIGS. 4 and 5, the absorbent article 110 can include an outer cover 26 having opaque regions 164, 170, 172, a first indicator region 166, and a second indicator region 168. The outer cover 126 can include a film layer 162 that includes a first indicator area 176 corresponding to the first indicator region 166 of the outer cover 126 and a second indicator area 178 corresponding to the second indicator region 168 of the outer cover 126. The outer cover 126 can also include opaque areas 174, 180, 182 corresponding to opaque regions 164, 170, 172 of the outer cover 126, respectively. Similar to the embodiment discussed above and illustrated in FIGS. 2 and 3, at least a portion of the first indicator region 166 in at least the crotch region 16 can be located between the first longitudinal edge 36 of the absorbent body 35 and the first coupling length 45 of where the containment flap 44 is bonded to the absorbent assembly 43 (in the embodiment illustrated in FIGS. 4 and 5, the first coupling length 45 is located where the bead of adhesive 49 bonds the containment flap 44 to the bodyside liner 28). Similarly, at least a portion of the second indicator region 168 in at least the crotch region 16 can be located between longitudinal edge 38 of the absorbent body 35 and the second coupling length 47 where the containment flap 46 is bonded to the absorbent assembly 43 (in the embodiment illustrated in FIGS. 4 and 5, the second coupling length 47 is located where the bead of adhesive 51 bonds the containment flap 46 to the bodyside liner 28). In the embodiment illustrated in FIGS. 4 and 5, the first indicator region 166 extends between the longitudinal edge 36 of the absorbent body 35 and the first coupling length 45 in the crotch region 16, as well as in the front and rear waist regions, 12, 14. The second indicator region 168 similarly extends between the longitudinal edge 38 of the absorbent body 35 and the second coupling length 45 in the crotch region 16, as well as in the front and rear waist regions 12, 14. Similar to the embodiment shown in FIGS. 2 and 3, the embodiment of the absorbent article 110 illustrated in FIGS. 4 and 5 can have the first indicator area 176 and the second indicator area 178 extend from the front waist edge 22 to the rear waist edge 24 of the absorbent article 110, and thus, the first and second indicator regions 166, 168 can extend from the front waist edge 22 to the rear waist edge 24.

As illustrated in FIGS. 4 and 5, the first indicator region 166 can extend laterally outside of the first coupling length 45 of containment flap 44 and the second indicator region 168 can extend laterally outside of the second coupling length 47 of containment flap 46 in at least one of the front waist region 12, the rear waist region 14, and the crotch region 16. In other words, the outer longitudinal edge 166b of the first indicator region 166 can be located laterally outside of the first coupling length 45 (i.e., farther from the longitudinal axis 29 than the coupling length 45 is from the longitudinal axis 29) and the outer longitudinal edge 168b of the second indicator region 168 can be located laterally outside of the second coupling length 47 (i.e., farther from the longitudinal axis 29 than the coupling length 47 is from the longitudinal axis 29). Although not shown, it is contemplated that the first indicator region 166 could extend to the longitudinal edge 18 of the absorbent article 110 and the second indicator region 168 could extend to the longitudinal edge 20 of the absorbent article 110 in some embodiments.

Additionally or alternatively, the first indicator region 166 can extend laterally inside of the longitudinal edge 36 of the absorbent body 35 (i.e., closer to the longitudinal axis 29 than the longitudinal edge 36 is from the longitudinal axis 29) in at least one of the front waist region 12, the rear waist region 14, and the crotch region 16. Likewise, the second indicator region 168 can extend laterally inside of the longitudinal edge 38 of the absorbent body 35 (i.e., closer to the longitudinal axis 29 than the longitudinal edge 38 is from the longitudinal axis 29) in at least one of the front waist region 12, the rear waist region 14, and the crotch region 16. Unlike the embodiment depicted in FIGS. 2 and 3, the first and second indicator regions 166 and 168 of FIGS. 4 and 5 can extend laterally inside of the respective longitudinal edges 36, 38 of the absorbent body 35 in each of the front waist region 12, the crotch region 16, and the rear waist region 14. In other words, the inner longitudinal edge 166a can be closer to the longitudinal axis 29 than is the longitudinal edge 36 of the absorbent body 35 and the inner longitudinal edge 168a can be closer to the longitudinal axis 29 than is the longitudinal edge 38 of the absorbent body 35 in one or more of these regions 12, 14, 16 of the absorbent article 10.

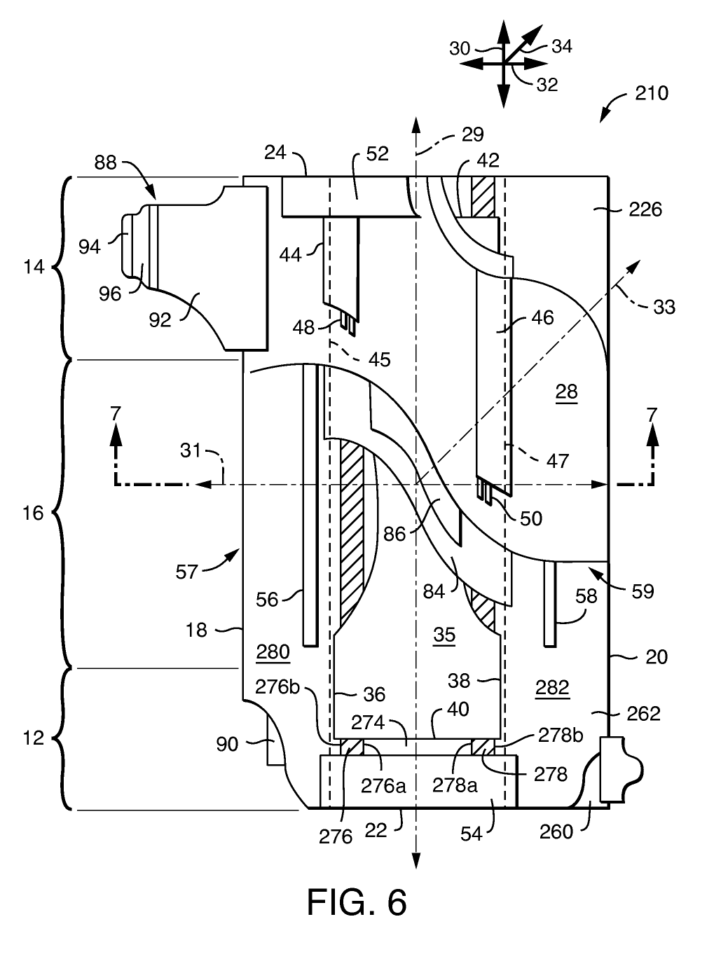

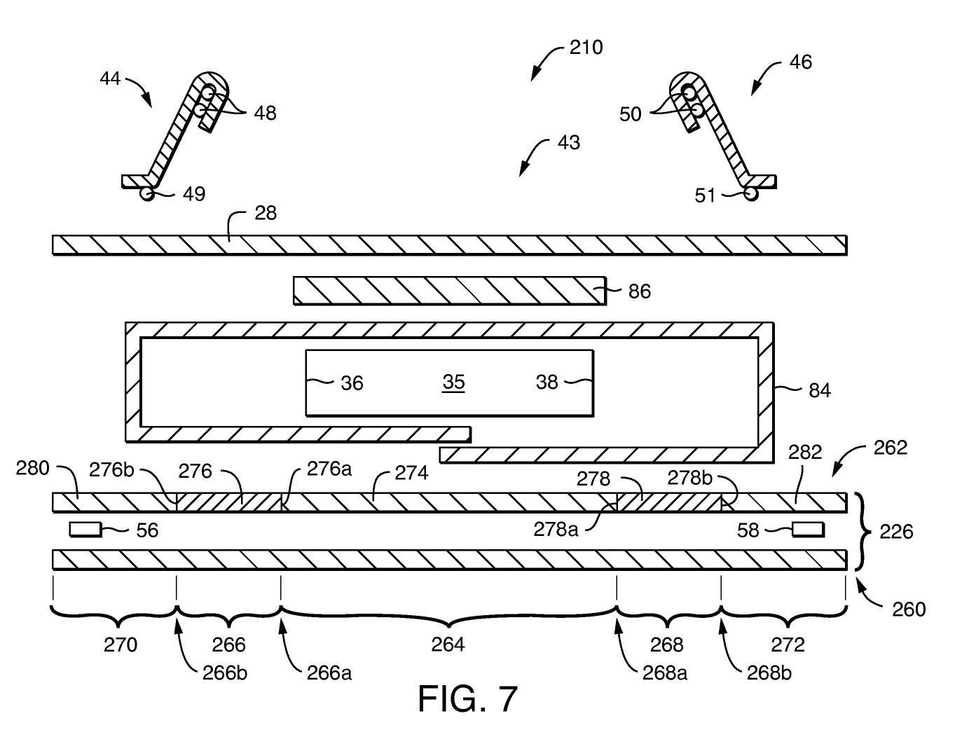

FIGS. 6 and 7 display another embodiment of an absorbent article 210 including an outer cover 226 having opaque regions 264, 270, 272, a first indicator region 266, and a second indicator region 268. The outer cover 226 can also include an inner layer 262 having a first indicator area 276 corresponding to the first indicator region 266, a second indicator area 278 corresponding to the second indicator region 268, and opaque indicator areas 274, 280, 282 corresponding to opaque regions 264, 270, 272, respectively. The first indicator region 266 and the second indicator region 268 of the outer cover 226, and thus the first indicator area 276 and the second indicator area 278 of the inner layer 262, can be configured similar to the first indicator region 66 and the second indicator region 68 of the outer cover 26, and thus the first indicator area 76 and the second indicator area 78 of inner layer 62, as described above with respect to article 10 and illustrated in FIGS. 2 and 3, except as noted below. Specifically, in the article 210 illustrated in FIGS. 6 and 7 the first indicator area 276 of the inner layer 262 of the outer cover 226, and thus the first indicator region 266 of the outer cover 226, is selectively located such that the outer longitudinal edge 276b of the first indicator area 276, and thus the outer longitudinal edge 266b of the first indicator region 266, is laterally inside of the longitudinal edge 36 of the absorbent body 35 in the front waist region 12, providing the entire lateral width of the first indicator area 276, and thus the first indicator region 266, being located laterally inside of the longitudinal edge 36 of the absorbent body 35 in the front waist region 12. The second indicator area 278 of the inner layer 262 of the outer cover 26, and thus the second indicator region 268 of the outer cover 26, is configured in a similar manner. Specifically, the outer longitudinal edge 278b of the second indicator area 278, and thus the outer longitudinal edge 268b of the second indicator region 268, is laterally inside of the longitudinal edge 38 of the absorbent body 35 in the front waist region 12, providing the entire lateral width of the second indicator area 278, and thus the second indicator region 268, being located laterally inside of the longitudinal edge 38 of the absorbent body 35 in the front waist region 12. Similar to absorbent articles 10, 110, described above, the absorbent article 210 can have the first indicator area 276 and the second indicator area 278, and thus the first indicator region 266 and the second indicator region 268, extend from the front waist edge 22 to the rear waist edge 24.

As discussed herein, a light transmittance of the indicator regions 66, 68, 166, 168, 266, 268 can be greater than a light transmittance of an opaque region 64, 70, 72, 164, 170, 172, 264, 270, 272 of the absorbent article 10, 110, 210. The light transmittance of the indicator regions 66, 68, 166, 168, 266, 268 as well as the light transmittance of the opaque regions 64, 70, 72, 164, 170, 172, 264, 270, 272 can be defined by the Light Transmittance Test method described further herein. The light transmittance of the indicator regions 66, 68, 166, 168, 266, 268 of the outer cover 26, 126, 226 can be affected by the light transmittance of the indicator areas 76, 78, 176, 178, 276, 278 of the inner layer 62, 162, 262 of the outer cover 26. Similarly, the light transmittance of the opaque regions 64, 70, 72, 164, 170, 172, 264, 270, 272 of the outer cover 26, 126, 226 can be affected by the light transmittance of the opaque areas 74, 80, 82, 174, 180, 182, 274, 280, 282 of the inner layer 62, 162, 262 of the outer cover 26. In some embodiments, a light transmittance of an opaque area 74, 80, 82, 174, 180, 182, 274, 280, 282 of the inner layer 62, 162, 262 of the outer cover 26, 126, 226 can range from about 5% to about 80% light transmittance, more preferably from about 20% to about 70% light transmittance, and most preferably from about 30% to about 60% light transmittance. A light transmittance of an indicator area 76, 78, 176, 178, 276, 278 of the inner layer 62, 162, 262 of the outer cover 26 can range from about 50% to about 99% light transmittance, more preferably from about 70% to about 97% light transmittance, and most preferably from about 80% to about 95% light transmittance. Of course, it is contemplated that other embodiments can have opaque areas and indicator areas of the inner layer of the outer cover with light transmittance values outside of the listed exemplary ranges. In some embodiments, the light transmittance of an indicator area 76, 78, 176, 178, 276, 278 of the inner layer 62, 162, 262 of the outer cover 26 can be 30% greater light transmittance than the light transmittance of an opaque area 74, 80, 82, 174, 180, 182, 274, 280, 282 of the inner layer 62, 162, 262 of the outer cover 26, 126, 226. In embodiments that include an outer cover that is only a single layer, such as a polymeric film similar to inner layer 62, 162, 262 described herein, the light transmittance of the indicator areas can be the light transmittance of the indicator regions and the light transmittance of the opaque areas can be the light transmittance of the opaque regions because the indicator areas are one in the same as the indicator regions and the opaque areas are one in the same as the opaque regions of the outer cover.

While the light transmittance of the indicator regions 66, 68, 166, 168, 266, 268 in the outer cover 26, and specifically in the inner layer 62, 162, 262 of the outer cover 26 in some embodiments, provide one factor in how well a caregiver will be able to see exudates, such as fecal material, through the indicator regions 66, 68, 166, 168, 266, 268 of the absorbent article 10, 110, 210, other factors can affect how well a caregiver will be able to see exudates through the indicator regions 66, 68, 166, 168, 266, 268. Other factors that can affect a caregiver's ability to see exudates in the absorbent article 10, 110, 210 can include the light transmittance of the outer layer 60, 160, 260 of the outer cover 26, 126, 226 if an outer layer 60, 160, 260 is present, the light transmittance of the bodyside liner 28, the opacity of a fluid transfer layer 84 if present, and the opacity of a fluid acquisition layer 86 if present.

The first indicator regions 66, 166, 266 and the second indicator regions 68, 168, 268 described in the embodiments above can provide the benefit of providing an indicator to a caregiver of whether the absorbent article 10 is retaining exudates. In particular, the first indicator regions 66, 166, 266 and the second indicator regions 68, 168, 268 can provide an indicator for fecal matter in the crotch region 16 of the absorbent articles 10, 110, 210. By selectively locating the first indicator regions 66, 166, 266 such that at least a portion of the first indicator regions 66, 166, 266 are between the longitudinal edge 36 of the absorbent body 35 and the first coupling length 45 of the containment flap 44 and at least a portion of the second indicator regions 68, 168, 268 are between the longitudinal edge 38 of the absorbent body 35 and the second coupling length 47 of the containment flap 46, the first indicator regions 66, 166, 266 and the second indicator regions 68, 168, 268 can each provide a viewpoint for the caregiver to determine whether fecal material is being retained in the article 10, 110, 210. This selective location near the longitudinal edges 36, 38 of the absorbent body 35 is especially beneficial in the circumstance that the article 10, 110, 210 is retaining low viscosity fecal material, which can be prevalent in younger children, as such fecal material is often difficult to absorb and may spread laterally towards the containment flaps 44, 46 of the absorbent article 10, 110, 210. A caregiver seeing such fecal material in the crotch region 16 of the absorbent article 10, 110, 210 through the indicator regions 66, 68, 166, 168, 266, 268 knows that the fecal material can be approaching and/or be at the leg containment flaps 44, 46, and thus, the absorbent article 10, 110, 210 can be changed while the fecal material is still contained by the absorbent article 10, 110, 210. Of course, it is to be noted that while preferred embodiments can include both a first indicator region 66, 166, 266 and a second indicator region 68, 168, 268 to provide such an indicator near each longitudinal edge 18, 20 of the absorbent article 10, 110, 210, some of these benefits can be realized with only a first indicator region 66, 166, 266.

Furthermore, by selectively locating the one or more indicator regions 66, 68, 166, 168, 266, 268 near the longitudinal edges 36, 38 of the absorbent body 35, the area of the indicator regions 66, 68, 166, 168, 266, 268 can be minimized. While caregivers often desire to know whether the absorbent article 10, 110, 210 is retaining exudates, such as fecal material, it is desirable to minimize the amount of exudates than can be seen through the absorbent article 10, 110, 210. The selective locations of the indicator regions 66, 68, 166, 168, 266, 268 described herein can provide the caregiver with an indicator or signal that the absorbent article 110 is retaining exudates, such as fecal material, but does so in a discrete manner without showing a majority of the absorbent structure that may also be containing exudates, such as urine.

Additionally, the selective location of the first indicator regions 66, 166, 266 and the second indicator regions 68, 168, 268 provide easy access for a caregiver to view the indicator regions 66, 68, 166, 168, 266, 268, even when the wearer is donning certain clothing over the absorbent article 10, 110, 210. For example, if the wearer is a child wearing clothing such as a onesie or shorts, the caregiver can easily slide their hand to move the clothing aside and view the first indicator region 66, 166, 266 on one side of the absorbent article 10, 110, 210 and/or the second indicator region 68, 168, 268 on the other side of the absorbent article 10, 110, 210 to visually inspect whether the article 10, 110, 210 is retaining exudates. This provides an advantage to the caregiver over articles that have exudate indicators that are aligned with the longitudinal axis 29 of the absorbent article, which may require a much more significant portion of the wearer's clothing to be moved, or completely removed, to check such indicator.

Yet another advantage that the indicator regions 66, 68, 166, 168, 266, 268 described herein can provide a caregiver include a dual purpose exudate indicator. For example, in embodiments that have the indicator regions 66, 68, 166, 168, 266, 268 that are present in the crotch region 16 as well as the front waist region 12, the indicator regions 66, 68, 166, 168, 266, 268 can provide an indication or signal to the caregiver of the presence of fecal material as well as urine. In some circumstances, fecal material may be retained by the absorbent article 10, 110, 210 in the crotch region 16 whereas urine may be retained by the absorbent article in both the crotch region 16 and the front waist region 12. By having at least one indicator region 66, 68, 166, 168, 266, 268 that is located in the front waist region 12 as well as the crotch region 16, the indicator region can provide the caregiver with an indication or signal of the presence of both fecal material and/or urine.

As referred to in the embodiments described herein and illustrated in FIGS. 1-7, absorbent articles 10, 110, 210 can have an outer cover 26, 126, 226 with at least one opaque region 64, 164, 264 and at least one indicator region 66, 166, 68, 168, 266, 268, with the at least one indicator region 66, 166, 68, 168, 266, 268 having a light transmittance that is greater than a light transmittance of the at least one opaque region 64, 164, 264. The embodiments discussed herein provide for such an absorbent article 10, 110, 210 due to configuration of the inner layer 62, 162, 262 in the outer cover 26, 126, 226 that can be comprised of a polymeric film (which can form the outer cover 26, 126, 226 itself or be one layer of the outer cover 26, 126, 226), however, the present disclosure is not limited to such a configuration. The present disclosure is intended to encompass absorbent articles with at least one opaque region 64, 164, 264 and at least one indicator region 66, 166, 68, 168, 266, 268, with the at least one indicator region 66, 166, 68, 168, 266, 268 having a light transmittance that is greater than a light transmittance of the at least one opaque region 64, 164, 264, where the opaque region 64, 164, 264 and the indicator region 66, 166, 68, 168, 266, 268 are configured based on selectively designed variations in light transmittance in an outer layer 60, 160, 260 of the outer cover 26, 126, 226, or based on selectively designed variations in light transmittance in an outer layer 60, 160, 260 and in an inner layer 62, 162, 262 of the outer cover 26, 126, 226. For example, an outer cover 26 could be configured such that the outer layer 60 of the outer cover 26 varied in basis weight or composition to vary the light transmittance between an opaque region 64 and an indicator region(s) 66, 68 and the inner layer 62 could be a uniform structure having substantially similar light transmittance properties throughout its construction.

An exemplary method for manufacturing an outer cover 26 for the absorbent article 10, 110, 210 will now be described. In one exemplary method, an absorbent article 10, 110, 210 with at least one opaque region 64, 164, 264 and at least one indicator region 66, 166, 68, 168, 266, 268, with the at least one indicator region 66, 166, 68, 168, 266, 268 having a light transmittance that is greater than a light transmittance of the at least one opaque region 64, 164, 264 can be manufactured by coextruding polymers using an extrusion die that can produce segmented films. For example, the inner layer 62, 162, 262 of FIGS. 3, 5, and 7, respectively, has several distinct adjacent areas, or segments. Referring to FIG. 3 and looking at the composition of the inner layer 62 from left to right, the inner layer 62 includes opaque area 80 which is adjacent to first indicator area 76, which in turn is adjacent opaque area 74, which in turn is adjacent to second indicator area 78, which in turn is adjacent to opaque area 82. Thus, in this embodiment, the inner layer 62 comprises five adjacent areas 80, 76, 74, 78, 82.

Such an inner layer 62, as well as inner layers 162 and 262 illustrated in FIGS. 5 and 7, can be manufactured by coextruding respecting adjacent segments such as, for example, using the method and apparatus for forming films as disclosed in U.S. Pat. No. 4,533,510 to Nissel et al., the entire contents of which are incorporated herein by reference. Extrusion dies for forming side-by-side coextruded films are commercially available from Extrusion Dies Industries, LLC of Chippewa Falls, Wis. and Cloeren Inc. of Orange, Tex. The desired components of the respective film segments can be separately mixed, heated and then coextruded into the respective side-by-side segments of a unitary segmented film. The film may be made by any one of a variety of film forming processes known in the art such as, for example, by using either cast or blown film equipment. Desirably, the first and second compositions are introduced to a segmented feed block prior to entering the film die. The segmented feed block allows contact between the first and second compositions prior to the compositions entering the film die. This early contact between the first and second compositions is believed to result in the extended overlap of the first and second segments at the interface between the segments. In one embodiment, a feed block suitable for making layered films from one or more polymer compositions is rotated 90 degrees with respect to the die body to provide segmented films rather than layered films. Suitable feed blocks, such as micro feed blocks or modular feed blocks, may be obtained from Randcastle Extrusion Systems, Inc., of Cedar Grove, N.J. The extruded segmented film is then processed as desired.

Furthermore, the exemplary method of manufacturing an outer cover 26, 126, 226 for the absorbent article 10, 110, 210 as discussed herein includes selection of materials into the segmented feed block to provide the desired opacity for opaque areas, such as opaque areas 80, 74, and 82, and the desired opacity for the indicator areas, such as first indicator area 74 and second indicator area 76. A wide variety of thermoplastic polymers can be used that are suitable for film formulation of the inner layer 62, 162, 262 of the outer cover 26, 126, 226 of the absorbent articles 10, 110, 210. Film forming polymers suitable for use with the present invention, alone or in combination with other polymers, include, by way of example only, polyolefins, stereoblock polymers, ethylene vinyl acetate (EVA), ethylene ethyl acrylate (EEA), ethylene acrylic acid (EAA), ethylene methyl acrylate (EMA), ethylene normal butyl acrylate (EnBA), polyester, polyethylene terephthalate (PET), nylon, ethylene vinyl alcohol (EVOH), polystyrene (PS), polyurethane (PU), polybutylene (PB), polyether esters, polyether amides, and polybutylene terephthalate (PBT).

A wide variety of polyolefin polymers exist and the particular composition of the polyolefin polymer and/or method of making the same is not believed critical to the present disclosure and thus both conventional and non-conventional polyolefins capable of forming films are believed suitable for use in the present disclosure. As used herein, “conventional” polyolefins refers to those made by traditional catalysts such as, for example, Ziegler-Natta catalysts. Suitable polyethylene and polypropylene polymers are widely available and, as one example, linear low density polyethylene is available from The Dow Chemical Company of Midland, Mich. under the trade name AFFINITY and conventional polypropylene is available from ExxonMobil Chemical Company of Houston, Tex. In addition, elastic and inelastic polyolefins made by “metallocene”, “constrained geometry” or “single-site” catalysts are also suitable for use in the present invention. Examples of such catalysts and polymers are described in U.S. Pat. No. 5,472,775 to Obijeski et al.; U.S. Pat. No. 5,451,450 to Erderly et al.; U.S. Pat. No. 5,278,272 to Lai et al.; U.S. Pat. No. 5,272,236 to Lai et al.; U.S. Pat. No. 5,204,429 to Kaminsky et al.; U.S. Pat. No. 5,539,124 to Etherton et al.; and U.S. Pat. No. 5,554,775 to Krishnamurti et al.; the entire contents of which are incorporated herein by reference. The aforesaid patents to Obijeski and Lai teach exemplary polyolefin elastomers and, in addition, exemplary low density polyethylene elastomers are commercially available from The Dow Chemical Company under the trade name AFFINITY, from ExxonMobil Chemical Company, under the trade name EXACT, and from Dupont Dow Elastomers, L.L.C. under the trade name ENGAGE. Moreover, exemplary propylene-ethylene copolymer plastomers and elastomers are commercially available from The Dow Chemical Company under the trade name VERSIFY and ExxonMobil Chemical Company under the trade name VISTAMAXX.

In addition, stereoblock polymers are also believed well suited for practicing embodiments of the present disclosure. The term “stereoblock polymer” refers to polymeric materials with controlled regional tacticity or stereosequencing to achieve desired polymer crystallinity. By controlling the stereoregularity during polymerization, it is possible to achieve atacticisotactic stereo blocks. Methods of forming polyolefin stereoblock polymers are known in the art and are described in the following articles: G. Coates and R. Waymouth, “Oscillating Stereocontrol: A Strategy for the Synthesis of Thermoplastic Elastomeric Polypropylene” 267 Science 217-219 (January 1995); K. Wagener, “Oscillating Catalysts: A New Twist for Plastics” 267 Science 191 (January 1995). Stereoblock polymers and methods of their production are also described in U.S. Pat. No. 5,594,080 to Waymouth et al., U.S. Pat. No. 5,208,304 to Waymouth and European Patent Application Publication No. 0475306 A1. As indicated above, by controlling the crystallinity of olefins it is possible to provide polymers exhibiting unique tensile modulus and/or elongation properties. In addition, polyolefin copolymers, and in particular propylene-ethylene copolymers, are also suitable for use with the present disclosure. As but one example, exemplary propylene-ethylene copolymers can comprise multi-step reactor products wherein an amorphous ethylene propylene random copolymer is molecularly dispersed in a predominately semi-crystalline high propylene monomer/low ethylene monomer continuous matrix. Examples of such polymers are described in U.S. Pat. No. 5,300,365 to Ogale; U.S. Pat. No. 5,212,246 to Ogale and U.S. Pat. No. 5,331,047 to Giacobbe. Such polymers are commercially available from LyondellBasell under the trade name CATALLOY polymers.

Other exemplary commercially available elastomeric materials include, but are not limited to, the following polymers: elastomeric polyurethanes such as, for example, those available under the trade name ESTANE from Lubrizol Advanced Materials, Inc.; elastomeric polyether esters such as, for example, those available under the trade name HYTREL from E. I. DuPont De Nemours & Company, and those available under the trade name ARNITEL available from DSM of Sittard, Holland (formerly available from Akzo Plastics of Arnhem, Holland); and elastomeric polyether amides commercially available from Arkema, Inc. under the trade name PEBAX. As additional examples, suitable thermoplastic elastomers also include those made from block copolymers having the general formula A-B-A′ where A and A′ are each a thermoplastic polymer endblock which contains a styrenic moiety such as a poly(vinyl arene) and where B is an elastomeric polymer midblock such as a conjugated diene or a lower alkene polymer. Further, exemplary block copolymers include A-B-A-B tetrablock polymers having an isoprene monomer unit hydrogenated to a substantially poly(ethylene-propylene) monomer unit such as a styrene-poly(ethylene-propylene)-styrene-poly(ethylene-propylene) elastomeric block copolymer. Examples of such styrene-olefin block copolymers include styrene-(ethylene-butylene), styrene-(ethylene-propylene), styrene-(ethylene-butylene)-styrene, styrene-(ethylene-propylene)-styrene, styrene-(ethylene-butylene)-styrene-(ethylene-butylene), styrene-(ethylene-propylene)-styrene-(ethylene-propylene), and styrene-ethylene-(ethylene-propylene)-styrene. These block copolymers may have a linear, radial or star-shaped molecular configuration. As specific examples, exemplary elastomers can comprise (polystyrene/poly(ethylene-butylene)/polystyrene) block copolymers available from the Kraton Polymers LLC under the trade name KRATON as well as polyolefin/KRATON blends such as those described in U.S. Pat. Nos. 4,663,220, 4,323,534, 4,834,738, 5,093,422, 5,304,599, and 5,332,613, the entire contents of the aforesaid references are incorporated herein by reference. Still other suitable copolymers include the S-I-S and S-B-S elastomeric copolymers available from Dexco Polymers of Houston, Tex. under the trade designation VECTOR®.

Other additives may be added in the manufacturing process by providing such additives in the feed block. Additives that can be used include melt stabilizers, crosslinking catalysts, pro-rad additives, processing stabilizers, heat stabilizers, light stabilizers, antioxidants, heat aging stabilizers, whitening agents, antiblocking agents, bonding agents, tackifiers, viscosity modifiers, etc. Examples of suitable tackifier resins may include, for instance, hydrogenated hydrocarbon resins. REGALREZ™ hydrocarbon resins are examples of such hydrogenated hydrocarbon resins, and are available from Eastman Chemical. Other tackifiers are available from ExxonMobil under the ESCOREZ™ designation. Viscosity modifiers may also be employed, such as polyethylene wax (e.g., EPOLENE™ C-10 from Eastman Chemical). Phosphite stabilizers (e.g., IRGAFOS available from Ciba Specialty Chemicals of Terrytown, New York and DOVERPHOS available from Dover Chemical Corp. of Dover, Ohio) are exemplary melt stabilizers. In addition, hindered amine stabilizers (e.g., CHIMASSORB available from Ciba Specialty Chemicals) are exemplary heat and light stabilizers. Further, hindered phenols are commonly used as an antioxidant in the production of films. Some suitable hindered phenols include those available from Ciba Specialty Chemicals of under the trade name “Irganox®”, such as Irganox® 1076, 1010, or E 201. Moreover, bonding agents may also be added to the film to facilitate bonding of the film to additional materials (e.g., nonwoven web). Typically, such additives (e.g., tackifier, antioxidant, stabilizer, etc.) are each present in an amount from about 0.001 wt. % to about 25 wt. %, in some embodiments, from about 0.005 wt. % to about 20 wt. %, and in some embodiments, from 0.01 wt. % to about 15 wt. % of the film.

Importantly, varying levels of opacifying filler materials can be added to the different segments of the segmented feed block to vary the light transmittance between different areas 80, 76, 74, 78, 82 of the inner layer 62 of the outer cover 26. Specifically, opacifying filler materials can be added in greater amounts or percentages to the corresponding segments of the segmented feed block that are extruded to form the opaque areas 80, 74, 82 of the inner layer 62 of the outer cover 26 in comparison to the amount of opacifying filler materials that are added to the corresponding segments of the segmented feed block that are extruded to form the indicator areas 76, 78 of the inner layer 62 of the outer cover 26. In some embodiments, no opacifying filler materials are added to the corresponding segments of the segmented feed block that are extruded to form the indicator areas 76, 78 of the inner layer 62 of the outer cover 26, in an attempt to increase the light transmittance of the indicator areas 76, 78 as much as possible. In other embodiments, opacifying filler materials can still be added to the corresponding segments of the segmented feed block that are extruded to form the indicator areas 76, 78 of the inner layer 62 of the outer cover 26, and thus, comprise a part of the indicator regions 66, 68 of the outer cover 26. Exemplary materials that can be used as opacifying filler materials include particles of calcium carbonate (CaCO₃), particles of titanium dioxide (TiO₂), particles of zinc oxide (ZnO), and pigmented particles. Of course, other opacifying filler materials can be utilized in various other embodiments.

The raw materials for the various segmented areas of the film are prepared and added to hoppers of an extrusion apparatus for each segmented area. It is to be noted that the same polymer materials can be used to compose both the opaque areas 74, 80, 82, 174, 180, 182, 274, 280, 282 and the indicator areas 74, 76, 174, 176, 274, 276 of the inner layer 62, 162, 262 of the outer cover 26, 126, 226. Alternatively, different polymer materials can be used to compose the opaque areas 74, 80, 82, 174, 180, 182, 274, 280, 282 and the indicator areas 74, 76, 174, 176, 274, 276 of the inner layer 62, 162, 262 of the outer cover 26, 126, 226. In one example, thermoplastic polymer particles, such as polyethylene particles, and an opacifying filler material, such as calcium carbonate particles, can be added to the hoppers corresponding to the segmented feed block for the opaque areas 80, 74, 82 of the film. It is to be noted that the same or different amounts of the opacifying filler material can be added to the corresponding feed block for the opaque areas 80, 74, 82, depending on whether it is desired to have substantially similar light transmittance in the opaque areas 80, 74, 82 in the film or different light transmittance between at least two or more of the opaque areas 80, 74, 82. The thermoplastic polymer particles, such as polyethylene particles, can be added to the hoppers corresponding to the segmented feed block for the indicator areas 76, 78 of the film. In some embodiments, no opacifying filler material is added to the segmented feed block for the indicator areas 76, 78 of the film, however, in other embodiments, some amount of opacifying filler material can be added to the segmented feed block for the indicator areas 76, 78 of the film, but in lesser amounts than at least one of the feed blocks corresponding to the opaque areas 80, 74, 82.

The materials are then dispersively mixed in the melt and compounded using any known technique, such as batch and/or continuous compounding techniques that employ, for example, a Banbury mixer, Farrel continuous mixer, single screw extruder, twin screw extruder, etc. Any known technique may be used to form a segmented film from the compounded materials, including blowing, casting, flat die extruding, etc. For example, the extruded film may be blown into nip rolls to form a single-layered precursor segmented film. The rolls may be kept at temperature sufficient to solidify and quench the precursor segmented film as it is formed, such as from about 20 to 60° C.

In one embodiment, the thermoplastic material and the opacifying filler material can be extruded through an extrusion die to form a first opaque area 80, a second opaque area 74, and a third opaque area 82 in the film. The first polymer material can also be coextruded through the extrusion die to form a first indicator area 76, the first indicator area 76 being formed between first opaque area 80 and second opaque area 74 and including a light transmittance that is greater than a light transmittance of the first opaque area 80 and greater than a light transmittance of the second opaque area 74. A second polymer material, which can be the same or different than the first polymer material, can also be coextruded through the extrusion die to form a second indicator area 78 that is located between the second opaque area 74 and the third opaque area 82 and including a light transmittance that is greater than a light transmittance of the second opaque area 74 and the third opaque area 82.

The segmented film may, optionally, be laminated to one or more additional films and/or fabrics. For example, a segmented film described herein may be bonded to a nonwoven web that forms an outer layer 60 of the outer cover 26 of the absorbent article 10.

Although thicker segmented films are possible, the unitary segmented film desirably has a maximum film thickness less than about 50 microns, desirably has a maximum film thickness between about 5 microns and about 50 microns, and more desirably has a maximum film thickness between about 10 microns and about 35 microns. Advantageously, the method described herein is capable of producing a film for an outer cover that is substantially continuous. This provides a more efficient and robust manufacturing method for an outer cover with one or more indicator regions as compared to other methods that involve coupling different materials together. Another benefit of the exemplary method of manufacturing such a film for an outer cover is that a thickness of the opaque areas of the film and a thickness of the first indicator area and, if present, the second indicator area of the film, are substantially similar. The “thickness” of the film forming the inner layer 62, 162, 262 is to be measured in the vertical direction 34. This provides the benefit of contributing towards a smooth outer cover 26, 126, 226 for the absorbent article 10, 110, 210 by preventing ridges or topography changes where thicknesses in outer cover materials change.

Absorbent Body:

The absorbent body 35 can be suitably constructed to be generally compressible, conformable, pliable, non-irritating to the wearer's skin and capable of absorbing and retaining liquid body exudates. The absorbent body 35 can be manufactured in a wide variety of sizes and shapes (for example, rectangular, trapezoidal, T-shape, I-shape, hourglass shape, etc.) and from a wide variety of materials. The size and the absorbent capacity of the absorbent body 35 should be compatible with the size of the intended wearer (infants to adults) and the liquid loading imparted by the intended use of the absorbent article 10. The absorbent body 35 can have a length and width that can be less than or equal to the length and width of the absorbent article 10. The absorbent body 35 can have two surfaces such as a wearer facing surface 64 and a garment facing surface (not shown). Edges, such as longitudinal side edges, 36 and 38, and such as front and back end edges, 40 and 42, can connect the two surfaces, 64 and 66.

In an embodiment, the absorbent body 35 can be composed of a web material of hydrophilic fibers, cellulosic fibers (e.g., wood pulp fibers), natural fibers, synthetic fibers, woven or nonwoven sheets, scrim netting or other stabilizing structures, superabsorbent material, binder materials, surfactants, selected hydrophobic and hydrophilic materials, pigments, lotions, odor control agents or the like, as well as combinations thereof. In an embodiment, the absorbent body 35 can be a matrix of cellulosic fluff and superabsorbent material. In an embodiment, the absorbent body 35 may be constructed of a single layer of materials, or in the alternative, may be constructed of two or more layers of materials.

Various types of wettable, hydrophilic fibers can be used in the absorbent body 35. Examples of suitable fibers include natural fibers, cellulosic fibers, synthetic fibers composed of cellulose or cellulose derivatives, such as rayon fibers; inorganic fibers composed of an inherently wettable material, such as glass fibers; synthetic fibers made from inherently wettable thermoplastic polymers, such as particular polyester or polyamide fibers, or composed of nonwettable thermoplastic polymers, such as polyolefin fibers which have been hydrophilized by suitable means. The fibers may be hydrophilized, for example, by treatment with a surfactant, treatment with silica, treatment with a material which has a suitable hydrophilic moiety and is not readily removed from the fiber, or by sheathing the nonwettable, hydrophobic fiber with a hydrophilic polymer during or after formation of the fiber. Suitable superabsorbent materials can be selected from natural, synthetic, and modified natural polymers and materials. The superabsorbent materials can be inorganic materials, such as silica gels, or organic compounds, such as cross-linked polymers. In an embodiment, the absorbent body 35 can be free of superabsorbent material.

The absorbent body 35 can be superposed over the inner layer 62 of the outer cover 26, extending laterally between the leg elastic members, 56, 58, and can be bonded to the inner layer 62 of the outer cover 26, such as by being bonded thereto with adhesive. However, it is to be understood that the absorbent body 35 may be in contact with, and not bonded with, the outer cover 26 and remain within the scope of this disclosure. In an embodiment, the outer cover 26 can be composed of a single layer and the absorbent body 35 can be in contact with the singer layer of the outer cover 26. In an embodiment, a layer, such as but not limited to, a fluid transfer layer 84, can be positioned between the absorbent body 35 and the outer cover 26.

Fluid Transfer Layer:

In various embodiments an absorbent article 10 can be constructed without a fluid transfer layer 84. In various embodiments the absorbent article 10 can have a fluid transfer layer 84. In an embodiment, the fluid transfer layer 84 can be in contact with the absorbent body 35. In an embodiment, the fluid transfer layer 84 can be bonded to the absorbent body 35. Bonding of the fluid transfer layer 84 to the absorbent body 35 can occur via any means known to one of ordinary skill, such as, but not limited to, adhesives. In an embodiment, a fluid transfer layer 84 can be positioned between the bodyside liner 28 and the absorbent body 35. In an embodiment, a fluid transfer layer 84 can completely encompass the absorbent body 35 and can be sealed to itself. In an embodiment a fluid transfer layer 84 may be composed of separate sheets of material which can be utilized to partially or fully encompass the absorbent body 35 and which can be sealed together using a sealing means such as, but not limited to, an ultrasonic bonder or other thermochemical bonding means or the use of an adhesive. In an embodiment, the fluid transfer layer 84 can be in contact with and/or bonded with the wearer facing surface 64 of the absorbent body 35.

The fluid transfer layer 84 can be pliable, less hydrophilic than the absorbent body 35, and sufficiently porous to thereby permit liquid body exudates to penetrate through the fluid transfer layer 84 to reach the absorbent body 35. In an embodiment, the fluid transfer layer 84 can have sufficient structural integrity to withstand wetting thereof and of the absorbent body 35. In an embodiment, the fluid transfer layer 84 can be constructed from a single layer of material or it may be a laminate constructed from two or more layers of material. In an embodiment, the fluid transfer layer 84 can include, but is not limited to, natural and synthetic fibers such as, but not limited to, polyester, polypropylene, acetate, nylon, polymeric materials, cellulosic materials and combinations thereof. In an embodiment, a fluid transfer layer 84 can include spunbond and/or meltblown materials. In an embodiment, the fluid transfer layer 84 can be a laminate of a meltblown nonwoven material having fine fibers laminated to at least one spunbond nonwoven material layer having coarse fibers. In such an embodiment, the fluid transfer layer 84 can be a spunbond-meltblown (“SM”) material, while in other embodiments, the fluid transfer layer 84 can be a spunbond-meltblown-spunbond (“SMS”) material. In various embodiments, the fluid transfer layer 84 can be hydrophilic. In various embodiments, the fluid transfer layer 84 can be hydrophobic and can be treated in any manner known in the art to be made hydrophilic. In an embodiment, the fluid transfer layer 84 can have a longitudinal length the same as, greater than, or less than the longitudinal length of the absorbent body 35.

Fluid Acquisition Layer:

In various embodiments the absorbent article 10 can have a fluid acquisition layer 86. The acquisition layer 86 can help decelerate and diffuse surges or gushes of liquid body exudates penetrating the bodyside liner 28. In an embodiment, the acquisition layer 86 can be positioned between the bodyside liner 28 and the outer cover 26. In an embodiment, the acquisition layer 86 can be positioned between the bodyside liner 28 and the absorbent body 35 to take in and distribute body exudates for absorption by the absorbent body 35. In an embodiment, the acquisition layer 86 can be positioned between the bodyside liner 28 and a fluid transfer layer 84 if a fluid transfer layer 84 is present.

In an embodiment, the acquisition layer 86 can be in contact with and/or bonded with the bodyside liner 28. In an embodiment in which the acquisition layer 86 is bonded with the bodyside liner 28, bonding of the acquisition layer 86 to the bodyside liner 28 can occur through the use of an adhesive and/or point fusion bonding, but is not limited to such methods of bonding. For example, the bodyside liner 28 could be bonded to the acquisition layer 86 by hydroentangling the bodyside liner 28 with the acquisition layer 86. The point fusion bonding can be selected from, but is not limited to, ultrasonic bonding, pressure bonding, thermal bonding, and combinations thereof. In an embodiment, the point fusion bonding can be provided in any pattern as deemed suitable.

The acquisition layer 86 can be rectangular in shape, hourglass in shape, or can be any other shape. The acquisition layer 86 may have any longitudinal length dimension as deemed suitable. For example, the acquisition layer 86 can have a longitudinal length shorter than, the same as, or longer than the longitudinal length of the absorbent body 35. In an embodiment, the acquisition layer 86 can have any length such that the acquisition layer 86 can be coterminous with the waist edges, 22 and 24, of the absorbent article 10. In an embodiment, the longitudinal length of the acquisition layer 86 can be the same as the longitudinal length of the absorbent body 35.

In an embodiment, the longitudinal length of the acquisition layer 86 can be shorter than the longitudinal length of the absorbent body 35. In such an embodiment, the acquisition layer 86 may be positioned at any desired location along the longitudinal length of the absorbent body 35. As an example of such an embodiment, the absorbent article 10 may contain a target area where repeated surges of bodily exudates typically occur in the absorbent article 10. The particular location of a target area can vary depending on the type of bodily exudate and/or on the age and gender of the wearer of the absorbent article 10. For example, a target area of the absorbent article 10 can vary based on urine or fecal matter. With respect to urination, males tend to urinate further toward the front waist region 12 of the absorbent article 10 and the target area may be phased forward within the absorbent article 10. For example, the target area for a male wearer may be positioned about 2¾″ forward of the longitudinal midpoint of the absorbent body 35 and may have a length of about ±3″ and a width of about ±2″. The female target area with respect to urination can be located closer to the center of the crotch region 16 of the absorbent article 10. For example, the target area for a female wearer may be positioned about 1″ forward of the longitudinal midpoint of the absorbent body 35 and may have a length of about ±3″ and a width of about ±2″. Thus, the relative longitudinal placement of the acquisition layer 86 within the absorbent article 10 can be selected to best correspond with the target area of either or both categories of wearers and can depend on what type of bodily exudate is specifically being targeted to control.

In an embodiment, the absorbent article 10 may contain a target area centered within the crotch region 16 of the absorbent article 10 with the premise that the absorbent article 10 would be worn by a female wearer. The acquisition layer 86, therefore, may be positioned along the longitudinal length of the absorbent article 10 such that the acquisition layer 86 can be substantially aligned with the target area of the absorbent article 10 intended for a female wearer. Alternatively, the absorbent article 10 may contain a target area positioned between the crotch region 16 and the front waist region 12 of the absorbent article 10 with the premise that the absorbent article 10 would be worn by a male wearer. The acquisition layer 86, therefore, may be positioned along the longitudinal length of the absorbent article 10 such that the acquisition layer 86 can be substantially aligned with the target area of the absorbent article 10 intended for a male wearer. In an embodiment, the acquisition layer 86 can have a size dimension that is the same size dimension as the target area of the absorbent article 10 or a size dimension greater than the size dimension of the target area of the absorbent article 10. In an embodiment, the acquisition layer 86 can be in contact with and/or bonded with the bodyside liner 28 at least partially in the target area of the absorbent article 10.

In various embodiments, the acquisition layer 86 can have a longitudinal length shorter than, the same as, or longer than the longitudinal length of the absorbent body 35. The acquisition layer 86 may have any width as desired. The width of the acquisition layer 86 may vary dependent upon the size and shape of the absorbent article 10 within which the acquisition layer 86 will be placed. The acquisition layer 86 can have a width smaller than, the same as, or larger than the width of the absorbent body 35. Within the crotch region 16 of the absorbent article 10, the acquisition layer 86 can have a width smaller than, the same as, or larger than the width of the absorbent body 35.

In exemplary embodiments, the acquisition layer 86 can include woven materials; fibrous nonwovens such as spunbond webs, meltblown webs and carded webs such as airlaid webs, bonded carded webs, and coform materials; binder and calendar bonded webs; foams, including open-cell foams; and scrim materials. The acquisition layer 86 can include various types of fibers such as natural fibers; cellulosic fibers; synthetic fibers composed of cellulose or cellulose derivatives, such as rayon fibers; inorganic fibers composed of an inherently wettable material, such as glass fibers; and synthetic fibers made from inherently wettable thermoplastic polymers, such as particular polyester or polyamide fibers, or nonwettable thermoplastic polymers, such as polyolefin fibers which have been hydrophilized by suitable means. The fibers may be hydrophilized, for example, by treatment with a surfactant, treatment with silica, treatment with a material which has a suitable hydrophilic moiety and is not readily removed from the fiber, or by sheathing the nonwettable, hydrophobic fiber with a hydrophilic polymer during or after formation of the fiber. In some embodiments, the acquisition layer 86 can be formed from a material that is substantially hydrophobic, such as a nonwoven web composed of polypropylene, polyethylene, polyester, and the like, and combinations thereof. In some embodiments, the acquisition layer 86 can include superabsorbent material. In some embodiments, the acquisition layer 86 can include materials having a basis weight ranging from about 10 gsm to about 300 gsm.

Bodyside Liner:

The bodyside liner 28 of the absorbent article 10 can overlay the absorbent body 35 and the outer cover 26 and can isolate the wearer's skin from liquid waste retained by the absorbent body 35. In various embodiments, a fluid transfer layer 84 can be positioned between the bodyside liner 28 and the absorbent body 35. In various embodiments, an acquisition layer 86 can be positioned between the bodyside liner 28 and the absorbent body 35 or a fluid transfer layer 84, if present. In various embodiments, the bodyside liner 28 can be bonded to the acquisition layer 86, or to the fluid transfer layer 84 if no acquisition layer 86 is present, via adhesive and/or by a point fusion bonding. The point fusion bonding may be selected from ultrasonic, thermal, pressure bonding, and combinations thereof.

In an embodiment, the bodyside liner 28 can extend beyond the absorbent body 35 and/or a fluid transfer layer 84, and/or an acquisition layer 86 to overlay a portion of the outer cover 26 and can be bonded thereto by any method deemed suitable, such as, for example, by being bonded thereto by adhesive, to substantially enclose the absorbent body 35 between the outer cover 26 and the bodyside liner 28. The bodyside liner 28 may be narrower than the outer cover 26, but it is to be understood that the bodyside liner 28 and the outer cover 26 may be of the same dimensions. It is also contemplated that the bodyside liner 28 may not extend beyond the absorbent body 35 and/or may not be secured to the outer cover 26. It is further contemplated that the bodyside liner 28 may be composed of more than one segment of material. The bodyside liner 28 can be of different shapes, including rectangular, hourglass, or any other shape. The bodyside liner 28 can be suitably compliant, soft feeling, and non-irritating to the wearer's skin and can be the same as or less hydrophilic than the absorbent body 35 to permit body exudates to readily penetrate through to the absorbent body 35 and provide a relatively dry surface to the wearer.

The bodyside liner 28 can be manufactured from a wide selection of materials, such as synthetic fibers (for example, polyester or polypropylene fibers), natural fibers (for example, wood or cotton fibers), a combination of natural and synthetic fibers, porous foams, reticulated foams, apertured plastic films, or the like. Examples of suitable materials include, but are not limited to, rayon, wood, cotton, polyester, polypropylene, polyethylene, nylon, or other heat-bondable fibers, polyolefins, such as, but not limited to, copolymers of polypropylene and polyethylene, linear low-density polyethylene, and aliphatic esters such as polylactic acid, finely perforated film webs, net materials, and the like, as well as combinations thereof.

Various woven and non-woven fabrics can be used for the bodyside liner 28. The bodyside liner 28 can include a woven fabric, a nonwoven fabric, a polymer film, a film-fabric laminate or the like, as well as combinations thereof. Examples of a nonwoven fabric can include spunbond fabric, meltblown fabric, coform fabric, carded web, bonded-carded web, bicomponent spunbond fabric, spunlace, or the like, as well as combinations thereof. The bodyside liner 28 need not be a unitary layer structure, and thus, can include more than one layer of fabrics, films, and/or webs, as well as combinations thereof. For example, the bodyside liner 28 can include a support layer and a projection layer that can be hydroentagled.

For example, the bodyside liner 28 can be composed of a meltblown or spunbond web of polyolefin fibers. Alternatively, the bodyside liner 28 can be a bonded-carded web composed of natural and/or synthetic fibers. The bodyside liner 28 can be composed of a substantially hydrophobic material, and the hydrophobic material can, optionally, be treated with a surfactant or otherwise processed to impart a desired level of wettability and hydrophilicity. The surfactant can be applied by any conventional means, such as spraying, printing, brush coating or the like. The surfactant can be applied to the entire bodyside liner 28 or it can be selectively applied to particular sections of the bodyside liner 28.

In an embodiment, a bodyside liner 28 can be constructed of a non-woven bicomponent web. The non-woven bicomponent web can be a spunbonded bicomponent web, or a bonded-carded bicomponent web. An example of a bicomponent staple fiber includes a polyethylene/polypropylene bicomponent fiber. In this particular bicomponent fiber, the polypropylene forms the core and the polyethylene forms the sheath of the fiber. Fibers having other orientations, such as multi-lobe, side-by-side, end-to-end may be used without departing from the scope of this disclosure. In an embodiment, a bodyside liner 28 can be a spunbond substrate with a basis weight from about 10 or 12 to about 15 or 20 gsm. In an embodiment, a bodyside liner 28 can be a 12 gsm spunbond-meltblown-spunbond substrate having 10% meltblown content applied between the two spunbond layers.

Although the outer cover 26 and bodyside liner 28 can include elastomeric materials, it is contemplated that the outer cover 26 and the bodyside liner 28 can be composed of materials which are generally non-elastomeric. In an embodiment, the bodyside liner 28 can be stretchable, and more suitably elastic. In an embodiment, the bodyside liner 28 can be suitably stretchable and more suitably elastic in at least the lateral or circumferential direction of the absorbent article 10. In other aspects, the bodyside liner 28 can be stretchable, and more suitably elastic, in both the lateral and the longitudinal directions 32, 30, respectively.

Containment Flaps:

In an embodiment, containment flaps, 44, 46, can be secured to the bodyside liner 28 of the absorbent article 10 in a generally parallel, spaced relation with each other laterally inward of the leg openings to provide a barrier against the flow of body exudates. In an embodiment, the containment flaps, 44, 46, can extend longitudinally from the front waist region 12 of the absorbent article 10, through the crotch region 16 to the back waist region 14 of the absorbent article 10. The containment flaps 44, 46 can be bonded to the bodyside liner 28 with beads of adhesive 49, 51, respectively, or other means as are known in the art. As previously discussed, each containment flap 44, 46 can alternatively be bonded to other components of the absorbent article 10 other than the bodyside liner 28, including, but not limited to, the outer cover 26.

The containment flaps, 44 and 46, can be constructed of a fibrous material which can be similar to the material forming the bodyside liner 28. Other conventional materials, such as polymer films, can also be employed. Each containment flap, 44 and 46, can include flap elastics, such as flap elastics 48 and 50, respectively. Suitable elastic materials for the flap elastic, 48 and 50, can include sheets, strands or ribbons of natural rubber, synthetic rubber, or thermoplastic elastomeric materials. The flap elastics, 48 and 50, as illustrated, can have two strands of elastomeric material extending longitudinally along the containment flaps, 44 and 46, in generally parallel, spaced relation with each other. The elastic strands can be within the containment flaps, 44 and 46, while in an elastically contractible condition such that contraction of the strands gathers and shortens the containment flaps, 44 and 46. As a result, the elastic strands can bias the containment flaps, 44 and 46, toward a position spaced from a position of where the containment flaps, 44 and 46, are bonded to absorbent article 10 such that a portion of the containment flaps, 44 and 46, can extend away from the bodyside liner 28 in a generally upright orientation of the containment flaps, 44 and 46, especially in the crotch region 16 of the absorbent article 10 when the absorbent article 10 is fitted on the wearer. For illustrative purposes, FIGS. 3, 5, and 7 show the containment flaps, 44 and 46, in such a generally upright orientation. The containment flaps, 44 and 46, can be connected to the flap elastics, 48 and 50, by partially doubling a portion of the containment flap, 44 and 46, material back upon itself by an amount which can be sufficient to enclose the flap elastics, 48 and 50, as is shown in FIGS. 3, 5, and 7. It is to be understood, however, that the containment flaps, 44 and 46, can have any number of strands of elastomeric material.

Leg Elastics:

Leg elastic members 56, 58 can be secured to the outer cover 26, such as by being bonded thereto by laminate adhesive, generally laterally inward of the longitudinal side edges, 18 and 20, of the absorbent article 10. The leg elastic members 56, 58 can form elasticized leg cuffs 57, 59, respectively, that further help to contain body exudates. In an embodiment, the leg elastic members 56, 58 may be disposed between the inner layer 62 and outer layer 60 of the outer cover 26 or between other layers of the absorbent article 10. The leg elastic members 56, 58 can be a single elastic member as illustrated in the figures herein, or each leg elastic member 56, 58 can include more than one elastic member. A wide variety of elastic materials may be used for the leg elastic members 56, 58. Suitable elastic materials can include sheets, strands or ribbons of natural rubber, synthetic rubber, or thermoplastic elastomeric materials. The elastic materials can be stretched and secured to a substrate, secured to a gathered substrate, or secured to a substrate and then elasticized or shrunk, for example, with the application of heat, such that the elastic retractive forces are imparted to the substrate.

Fastening System:

In an embodiment, the absorbent article 10 can include a fastener system. The fastener system can include one or more back fasteners 88 and one or more front fasteners 90. Portions of the fastener system may be included in the front waist region 12, back waist region 14, or both. The fastener system can be configured to secure the absorbent article 10 about the waist of the wearer and maintain the absorbent article 10 in place during use. In an embodiment, the back fasteners 88 can include one or more materials bonded together to form a composite ear as is known in the art. For example, the composite fastener may be composed of a stretch component 92, a nonwoven carrier or hook base 94, and a fastening component 96.

Waist Elastic Members:

In an embodiment, the absorbent article 10 can have waist elastic members, 52 and 54, which can be formed of any suitable elastic material. The waist elastic member 52 can be in a rear waist region 12 of the absorbent article 10 and the waist elastic member 54 can be in a front waist region 14 of the absorbent article 10. Suitable elastic materials for the waist elastic members 52, 54 can include, but are not limited to, sheets, strands or ribbons of natural rubber, synthetic rubber, or thermoplastic elastomeric polymers. The elastic materials can be stretched and bonded to a substrate, bonded to a gathered substrate, or bonded to a substrate and then elasticized or shrunk, for example, with the application of heat, such that elastic retractive forces are imparted to the substrate. It is to be understood, however, that the waist elastic members, 52 and 54, may be omitted from the absorbent article 10 without departing from the scope of this disclosure.

Light Transmittance Test:

As referred to in the embodiments herein, absorbent articles 10, 110, 210 can have an outer cover with at least one opaque region 64, 164, 264 and at least one indicator region 66, 166, 68, 168, 266, 268, with the at least one indicator region 66, 166, 68, 168, 266, 268 having a light transmittance that is greater than a light transmittance of the at least one opaque region 64, 164, 264. The embodiments discussed herein provide such an absorbent article 10, 110, 210 due to the configuration of an inner layer 62, 162, 262, such as a film, in the outer cover 26, 126, 226 (which can form the outer cover itself or function as an inner layer 62, 162, 262 of the outer cover). However, as noted above, the present disclosure is intended to encompass absorbent articles with at least one opaque region 64, 164, 264 and at least one indicator region 66, 166, 68, 168, 266, 268, with the at least one indicator region 66, 166, 68, 168, 266, 268 having a light transmittance that is greater than a light transmittance of the at least one opaque region 64, 164, 264, where the opaque region 64, 164, 264 and the indicator region 66, 166, 68, 168, 266, 268 are configured based on selectively designed variations in light transmittance in an outer layer 60, 160, 162 of the outer cover 26, 126, 226, or based on selectively designed variations in light transmittance in an outer layer 60, 160, 162 and an inner layer 62, 162, 262 of the outer cover 26, 126, 226. Regardless of the configuration used to achieve an outer cover 26, 126, 226 with at least one opaque region 64, 164, 264 and at least one indicator region 66, 166, 68, 168, 266, 268, with the at least one indicator region 66, 166, 68, 168, 266, 268 having a light transmittance that is greater than a light transmittance of the at least one opaque region 64, 164, 264, the following Light Transmittance Test can be used to determine the light transmittance for the at least one opaque region 64, 164, 264 and the at least one indicator region 66, 166, 68, 168, 266, 268 of the article.

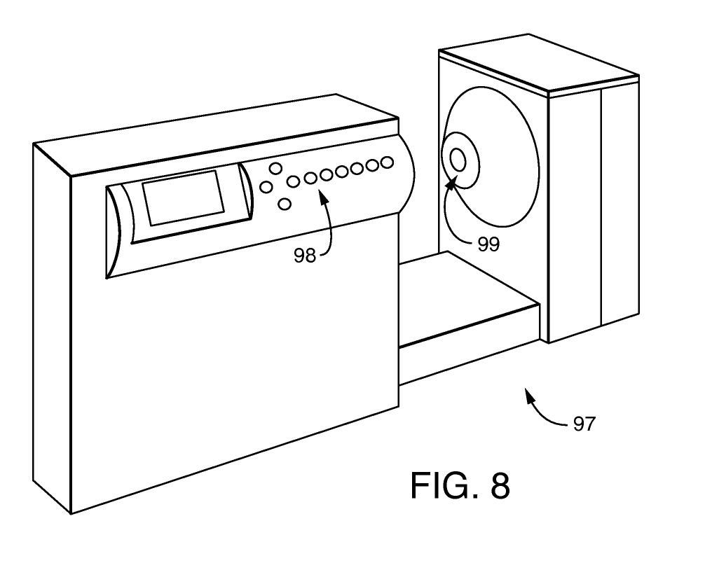

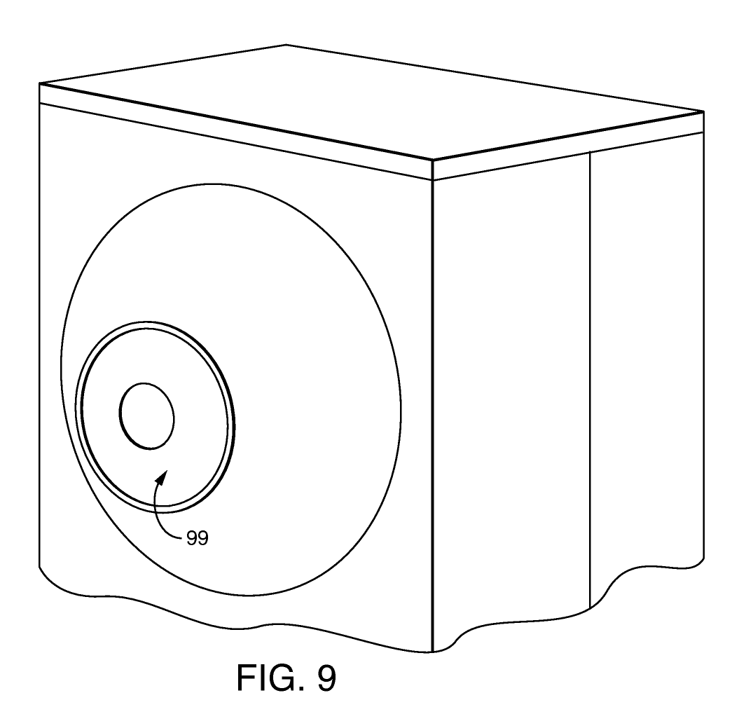

The Light Transmittance Test can be performed using a Haze Guard Plus Model #4725 manufactured by BYK-Gardner GmbH, such as the device 97 illustrated in FIG. 8. The Haze Guard Plus Model #4725 includes a control panel 98 and a sample port 99. The control panel 98 includes control buttons as well as a display. The sample port 99 is 25.4 mm in diameter. The Light Transmittance Testing should be conducted in a standard laboratory atmosphere 23±1° C. (73.4±1.8° F.) and 50±2% relative humidity.

For purposes of providing context for the Light Transmittance Test with respect to an exemplary embodiment, the Light Transmittance Test will be described herein for measuring the light transmittance for an indicator area 76, 78, 176, 178, 276, 278 and an opaque area 74, 174, 274 of a polymeric film which can form an inner layer 62, 162, 262 of the outer cover 26, 126, 226 (such as shown in FIGS. 1-7). In particular, the Light Transmittance Test discussed below will be directed towards one embodiment of an absorbent article 10 having an outer cover 26 with an inner layer 62 having indicator areas 76, 78 and the light transmittance for an opaque areas 74, 80, 82, such as the outer cover 26 of the absorbent article 10 of FIGS. 1-3. However, as noted above, the Light Transmittance Test can be conducted to measure the light transmittance of an opaque region 64, 164, 264 and an indicator region 66, 166, 68, 168, 266, 268 where the samples tested from each region are in laminate form, comprising an inner layer 62, 162, 262 and an outer layer 60, 160, 260 to each sample, or where the outer cover only includes a single layer, whether it is of a polymeric film, nonwoven, or other material.

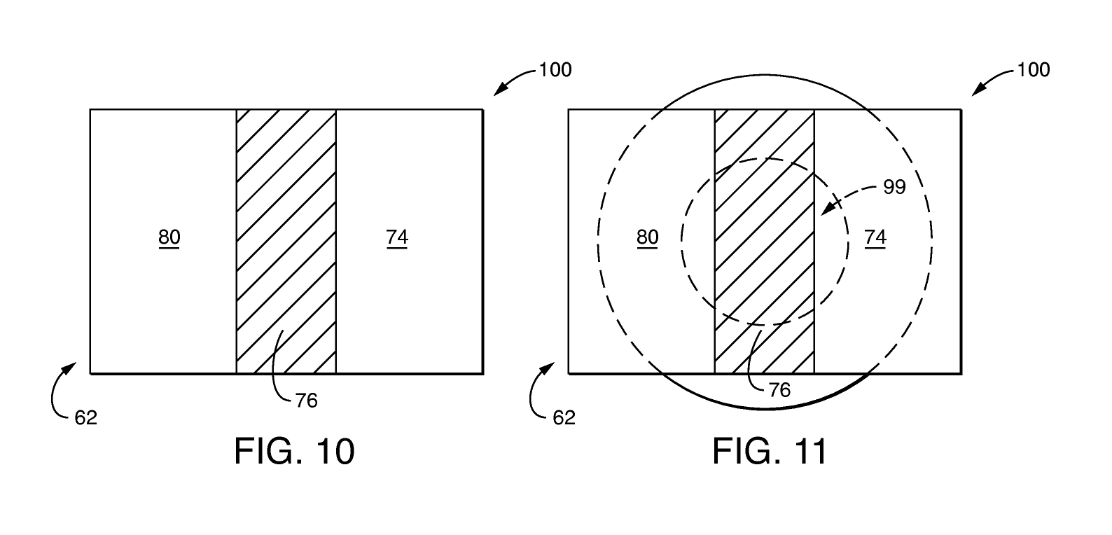

To measure the light transmittance of the indicator area 76 of the inner layer 62 and the light transmittance of one or more of the opaque areas 74, 80 of the inner layer 62 of the article depicted in FIGS. 1-3, the inner layer 62 is removed from all other components of the absorbent article 10, such as the portion of the sample 100 of the inner layer 62 shown in FIGS. 10 and 11. As will be noted further below, the size of the sample 100 should be prepared such that five (5) light transmittance values can be measured along the longitudinal length of the sample in the desired area to be tested, such that an average light transmission value can be obtained for that specific area of the inner layer 62. For example, when measuring the light transmittance of the indicator area 76 of the inner layer 62, sample 100 should be prepared such that five (5) light transmittance values can be taken and then averaged to provide the light transmittance value for the indicator area 76. The five (5) points of measuring the light transmittance should be taken from evenly spaced locations along the longitudinal length of the desired area to be tested, and should be selected at points that are substantially free from wrinkles and any printing of ink graphics.

To begin the Light Transmittance Test with the device 97, the Haze Guard Plus Model can be prepared by following these instructions:
1. Turn on the Haze Guard Plus Model device 97.
2. Wait for the self-diagnostics and warm up to be completed.
3. Validate the device 97 by pressing the down arrow on the control panel 98. In the set up menue, highlight calibrate by using the up and down arrows on the control panel 98.
4. Press operate and the control panel will provide a prompt to cover the sample port 99.
5. Cover the sample port 99 with the manufacturer-provided cover such that light will not be transmitted to the sample port 99.
6. Remove the cover from the sample port 99 when prompted by the device 97.
7. Place the manufacturer-provided clarity standard on the sample port 99 and press operate on the control panel 98.
8. The light transmittance value should be within ±1% of the value on the clarity standard.

Once the device 97 is prepared, samples 100 for the Light Transmittance Test can be tested for light transmittance in the desired areas. The light transmittance of a specific area of an inner layer or region of the outer cover can be measured by following these steps:
1. Place the desired area of the sample 100 to be tested over the sample port 99, ensuring that the sample 100 covers the entire sample port 99 and that there are no wrinkles in the sample 100 over the sample port 99.
2. Press the operate button on the control panel 98 and record the light transmittance value displayed on the control panel 98, which provides a percentage of the light transmitted through the sample 100 placed over the sample port 99.
3. Repeat steps 1 and 2 to provide four (4) more light transmittance values for the desired area of the sample 100, creating a total of five (5) light transmittance values along the longitudinal length of the desired area of the sample 100.
4. Average the five light transmittance values for the desired area to obtain the light transmittance value for the desired area.

In some embodiments, the sample 100 selected for Light Transmittance Testing may include an area of the inner layer or region of the outer cover that is desired to test for light transmittance but the desired area or region is narrower than the diameter of the sample port 99. For example, indicator area 76 of the inner layer 62 of the outer cover 26, in some embodiments, may have a lateral width that is less than 25.4 mm, the diameter of the sample port 99 (e.g., FIGS. 10 and 11 depict indicator area 76 being narrower than the sample port 99). In such a circumstance, when performing the Light Transmittance Testing on the indicator area 76 of the inner layer 62, the sample 100 should be aligned such that the indicator area 76 is generally aligned with the central axis of the sample port 99, as shown in FIG. 11, and the Light Transmittance Test can be completed following the steps outlined above. While such a measurement of light transmittance will not provide the exact light transmittance value for the indicator area 76 of the inner layer 62 (because a portion of the opaque areas 74, 80 are also within the diameter of the sample port 99), the measurement of light transmittance will provide a light transmittance value that can be compared with the light transmittance value when using the Light Transmittance Test to test the light transmittance of the opaque areas 74, 80 of the inner layer 62, which are wider than the diameter of the sample port 99. Thus, such a circumstance will still provide an assessment as to whether the indicator area 76 has a light transmittance that is greater than the light transmittance of the opaque areas 74, 80.

All documents cited in the Detailed Description of the Disclosure are, in relevant part, incorporated herein by reference; the citation of any document is not to be construed as an admission that it is prior art with respect to the present invention. To the extent that any meaning or definition of a term in this written document conflicts with any meaning or definition of the term in a document incorporated by references, the meaning or definition assigned to the term in this written document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

1. An absorbent article including a front waist region, a rear waist region, and a crotch region, the absorbent article comprising:
an absorbent assembly including a bodyside liner, an outer cover, and an absorbent body disposed between the bodyside liner and the outer cover, the absorbent body including a first longitudinal edge and a second longitudinal edge; and
a pair of containment flaps including a first containment flap and a second containment flap, the first containment flap being coupled to the absorbent assembly along a first coupling length, the second containment flap being coupled to the absorbent assembly along a second coupling length;
the outer cover including at least one opaque region and a first indicator region, the first indicator region including a light transmittance that is greater than a light transmittance of the at least one opaque region, at least a portion of the first indicator region being located between the first longitudinal edge of the absorbent body and the first coupling length of the first containment flap in at least the crotch region.

2. The absorbent article of claim 1, wherein the outer cover further includes a second indicator region, the second indicator region being separate from the first indicator region and including a light transmittance greater than the light transmittance of the at least one opaque region.

3. The absorbent article of claim 2, wherein the outer cover comprises a film, the film providing at least one opaque area corresponding to the at least one opaque region of the outer cover, a first indicator area corresponding to the first indicator region of the outer cover, and a second indicator area corresponding to the second indicator region of the outer cover, and wherein a light transmittance of the at least one opaque area of the film is from about 30% light transmittance to about 60% light transmittance and a light transmittance of the first indicator area of the film and a light transmittance of the second indicator area of the film are each from about 80% light transmittance to about 95% light transmittance.

4. The absorbent article of claim 2, wherein the outer cover comprises a film, the film providing at least one opaque area corresponding to the at least one opaque region of the outer cover, a first indicator area corresponding to the first indicator region of the outer cover, and a second indicator area corresponding to the second indicator region of the outer cover, wherein a light transmittance of the first indicator area of the film and a light transmittance of the second indicator area of the film are each at least about 30% light transmittance greater than a light transmittance of the at least one opaque area of the film.

5. An absorbent article including a front waist region, a rear waist region, and a crotch region, the absorbent article comprising:
an absorbent assembly comprising a bodyside liner, an absorbent body including a first longitudinal edge and a second longitudinal edge, and an outer cover, the absorbent body being disposed between the bodyside liner and the outer cover, the outer cover comprising a substantially continuous film including at least one opaque area corresponding to a first opaque region of the outer cover and a first indicator area corresponding to a first indicator region of the outer cover, the first indicator region including a light transmittance that is greater than a light transmittance of the at least one opaque region and being located in at least the crotch region, the outer cover being configured such that a thickness of the opaque area of the film and a thickness of the first indicator area of the film are substantially similar.

6. The absorbent article of claim 1, wherein the outer cover further includes a second indicator region, the second indicator region being separate from the first indicator region and having a light transmittance that is greater than the light transmittance of the at least one opaque region, wherein the at least one opaque region is disposed between the first indicator region and the second indicator region, and wherein at least a portion of the second indicator region is located between the second longitudinal edge of the absorbent body and the second coupling length of the second containment flap in the at least the crotch region.

7. The absorbent article of claim 1, wherein the first indicator region extends between the first longitudinal edge of the absorbent body and the first coupling length of the first containment flap in the crotch region.

8. The absorbent article of claim 7, wherein the outer cover further includes a second indicator region, the second indicator region being separate from the first indicator region and having a second light transmittance that is greater than the light transmittance of the at least one opaque region, wherein the at least one opaque region is disposed between the first indicator region and the second indicator region, and wherein the second indicator region extends between the second longitudinal edge of the absorbent body and the second coupling length of the second containment flap in the crotch region.

9. The absorbent article of claim 1, wherein the first indicator region extends from a front waist edge of the outer cover to a rear waist edge of the outer cover.

10. The absorbent article of claim 9, wherein the first indicator region is further configured such that the first indicator region extends laterally inside of the first longitudinal edge of the absorbent body in the front waist region.

11. The absorbent article of claim 1, wherein the first indicator region includes an inner longitudinal edge and an outer longitudinal edge, the inner longitudinal edge of the first indicator region is laterally outside of the first longitudinal edge of the absorbent body in the crotch region, and wherein the outer longitudinal edge of the first indicator region is laterally inside of the first longitudinal edge of the absorbent body in the front waist region.

12. The absorbent article of claim 5, wherein the thickness of the first indicator area of the film is from about 10 microns to about 50 microns.

13. The absorbent article of claim 5, wherein the first indicator area of the film does not include an opacifying filler material.

14. A method of manufacturing an outer cover for an absorbent article, the outer cover comprising a segmented film, the method comprising:
providing a first polymer material and a second polymer material;
providing an opacifying filler material;
providing an extrusion die;
extruding the first polymer material and the opacifying filler material through the extrusion die to form a first opaque area and a second opaque area; and
coextruding the second polymer material through the extrusion die to form a first indicator area between the first opaque area and the second opaque area to form the segmented film, the first indicator area including a light transmittance that is greater than a light transmittance of the first opaque area and greater than a light transmittance of the second opaque area.

15. The method of claim 14, further comprising:
coextruding the second polymer material through the extrusion die to form a second indicator area and extruding the first polymer material and the opacifying filler material through the extrusion die to form a third opaque area, the second indicator area being formed between the second opaque area and the third opaque area, the second indicator area including a light transmittance that is greater than the light transmittance of the first opaque area and greater than a light transmittance of the third opaque area.

16. The method of claim 14, further comprising:
laminating the segmented film to a non-woven layer.

17. The method of claim 14, wherein the first polymer material is the same as the second polymer material.

18. A method of manufacturing an absorbent article, the absorbent article comprising an outer cover manufactured pursuant to claim 15.

19. The method of manufacturing an absorbent article of claim 18, further comprising:
providing a bodyside liner and an absorbent body;
coupling the bodyside liner to the outer cover with the absorbent body disposed between the outer cover and the bodyside liner to form an absorbent assembly;
providing a pair of containment flaps including a first containment flap and a second containment flap;
coupling the first containment flap to the absorbent assembly along a first coupling length such that at least a portion of the first indicator area is disposed between a first longitudinal edge of the absorbent body and the first coupling length of the first containment flap in at least a crotch region of the absorbent article; and
coupling the second containment flap to the absorbent assembly along a second coupling length such that at least a portion of the second indicator area is disposed between a second longitudinal edge of the absorbent body and the second coupling length of the second containment flap in at least the crotch region of the absorbent article.
